(19) United States
(12) Patent Application Publication
(10) Pub. No.: US 2014/0031852 A1
(43) Pub. Date: Jan. 30, 2014

(54) Devices and methods for Dilating a Eustachian Tube

(71) Applicant: SINUSYS CORPORATION, (US)

(72) Inventors: David E. Edgren, Los Altos, CA (US); William Jason Fox, San Carlos, CA (US); Jerome E. Hester, Menlo Park, CA (US)

(73) Assignee: SINUSYS CORPORATION, Palo Alto, CA (US)

(21) Appl. No.: 13/777,654

(22) Filed: Feb. 26, 2013

Related U.S. Application Data
(60) Provisional application No. 61/604,970, filed on Feb. 29, 2012.

(51) Int. Cl. A61M 29/02

(52) U.S. Cl. CPC A61M 29/02
USPC 606/199

(57) ABSTRACT

Dilators are provided for insertion into, and dilation of, a Eustachian tube of an animal, e.g., a human being. The dilators may be placed in the Eustachian tube via the oral or nasal passageway, and the nasopharynx. The dilators are configured to be self-expanding and include a driver configured to expand an expandable portion from a non-expanded configuration to an expanded configuration. Also provided are kits that include the dilator, an insertion device for inserting the dilator, and methods for inserting the dilator into a Eustachian tube.

### CROSS-REFERENCE TO RELATED APPLICATIONS

This application claims priority benefit of U.S. provisional application Ser. No. 61/604,970, filed Feb. 29, 2012, which application is incorporated herein by reference.

This application is related to U.S. patent application Ser. Nos. 13/219,505 and 13/219,497, both filed Aug. 26, 2011, the disclosures of each of which are incorporated herein by reference.

### INTRODUCTION

The Eustachian tube is a hollow lined tube that connects the middle ear to the nasopharynx. Typically, the middle ear portion of the tube can only be accessed by incising the tympanic membrane (i.e., the eardrum) or ear canal skin. The nasal portion of the tube is surrounded by cartilage that regulates opening and closing actions (torus tubarius). In its resting state, the Eustachian tube is in the closed position. Eustachian tube opening action is mediated by contraction of surrounding muscles that impinge upon the tube and torus tubarius. An opened tube ventilates and drains the middle ear and maintains proper pressure relationships among the tympanic membrane, middle ear and nasopharynx.

Eustachian tube dysfunction has been implicated in the development of various otologic diseases. The etiology of acute otitis media is hypothesized to be due to bacteria traveling into the middle ear from the nasal cavity in a setting of inflammation, which prevents the middle ear from draining properly. Chronic otitis media occurs when the Eustachian tube fails to ventilate the middle ear over an extended period. In these cases, fluid and thickened mucous accumulate in the middle ear, causing hearing loss. As difficulty ventilating the middle ear continues, skin from the tympanic membrane may invaginate to form a cholesteatoma, and cause chronic infection and destruction of the ossicles, inner ear and mastoid air cell system.

Eustachian tube dysfunction is especially problematic for patients who are unable to clear their ears when flying and diving. In the setting of rapidly changing barometric conditions, as in flying and diving, inability to ventilate the middle ear sufficiently can lead to barotrauma with accumulation of fluid or blood in the middle ear. On occasion, Eustachian tube dysfunction patients can experience tympanic membrane rupture, deep hearing loss and dizziness.

Treatment of Eustachian tube dysfunction has mainly been directed at ventilation of the middle ear via the tympanic membrane. Typically, a myringotomy or incision through the substance of the tympanic membrane is created, and a ventilation tube is placed within the incision. These ventilation tubes or grommets have been commercially available for over 50 years. Such treatments are associated with numerous drawbacks. For example, ventilation tubes are typically spontaneous and uncontrollably extruded from the tympanic membrane about 4 to 9 months after placement. The invasiveness of surgical procedures to the tympanic membrane also represents a potential source of complications.

Myringotomy can lead to persistent abnormalities with the tympanic membrane. In the pediatric age group where myringotomy with ventilation tube insertion is a common procedure, the most concerning complication is permanent tympanic membrane perforation. When tympanic membranes heal after myringotomy, many are clearly abnormal, such as, formation of retraction pockets, thin atrophic membranes, and tympanosclerosis. The impact of these undesirable changes is hearing loss that ranges from 3-5 decibels (dB). Incision into the ear canal is a more technically complex surgical process. Bleeding in the ear canal and scarring of the tympanic membrane are common outcomes.

### SUMMARY

Dilators are provided for insertion into, and dilation of, a Eustachian tube of an animal, e.g., a human being. The dilators may be placed in the Eustachian tube via the oral or nasal passageway, and the nasopharynx. The dilators are configured to be self-expanding and include a driver configured to expand an expandable portion from a non-expanded configuration to an expanded configuration. Also provided are kits that include the dilator, an insertion device for inserting the dilator, and methods for inserting the dilator into an Eustachian tube.

Disclosed herein are devices for insertion into and dilation of a Eustachian tube. In some embodiments, the devices are used to affect pressure equilibration between the middle ear and ambient pressure and/or to allow drainage of the middle ear cavity.

Disclosed herein is a method for dilating a dysfunctional Eustachian tube of an animal, such as a human. The method can be used in one or more of several medical procedures, such as equalization of the pressure within a patient's middle ear with ambient (e.g., environmental) pressure and/or allowing a liquid, e.g., blood, puss, mucous, etc., to drain from a middle ear cavity of the animal.

Also disclosed herein is a dilator for dilating a dysfunctional Eustachian tube of an animal, such as a human, and an insertion device for inserting the dilator into the Eustachian tube. The dilator and insertion device can be used in one or more of several medical procedures, such as equalization of the pressure within a patient's middle ear with ambient (e.g., environmental) pressure and/or allowing a liquid, e.g., blood, puss, mucous, etc., to drain from a middle ear cavity of the animal.

In some embodiments, a method of dilating a dysfunctional Eustachian tube of an animal is provided. The method includes: inserting a self-expanding dilator through a nasopharyngeal opening of the Eustachian tube via an oral or nasal passageway of the animal, where the dilator is configured to be self-expanding after insertion and includes a driver configured to expand an expandable portion from a non-expanded configuration to an expanded configuration. The non-expanded configuration is sized to be positioned within the dysfunctional Eustachian tube and the expanded configuration has a size sufficient to dilate the Eustachian tube.

Embodiments of the method may include that the self-expanding dilator expands over a period of 0.5 hours or more.

Embodiments of the method may include that the self-expanding dilator expands over a period of 0.5 to 4 hours.

Embodiments of the method may include that the self-expanding dilator expands over a period of 0.5 to 2 hours.

Embodiments of the method may include that the driver includes an osmotically active agent.

Embodiments of the method may include that the driver includes an expandable polymeric matrix.

Embodiments of the method may include that the driver is configured to absorb water from the animal to expand the dilator.

Embodiments of the method may include removing the dilator after the expandable portion expands from the non-expanded configuration to the expanded configuration.

Embodiments of the method may include that the dilator is configured to equalize pressure within a middle ear of the animal with ambient pressure.

Embodiments of the method may include that the dilator includes a conduit configured to drain liquid from a middle ear cavity of the animal.

Embodiments of the method may include that the animal is a human.

In some embodiments, a dilator for dilating a dysfunctional Eustachian tube of an animal is provided. The dilator includes: a self-expanding dilator configured to expand an expandable portion from a non-expanded configuration to an expanded configuration, where the non-expanded configuration is sized to be inserted through a nasopharyngeal opening of the Eustachian tube via an oral or nasal passageway of the animal and positioned within the dysfunctional Eustachian tube. The dilator is configured to be self-expanding after insertion and includes a driver configured to expand an expandable portion from a non-expanded configuration to an expanded configuration, where the non-expanded configuration is sized to be positioned within the dysfunctional Eustachian tube and the expanded configuration has a size sufficient to dilate the Eustachian tube.

Embodiments of the dilator may include that the self-expanding dilator expands over a period of 0.5 hours or more.

Embodiments of the dilator may include that the self-expanding dilator expands over a period of 0.5 to 4 hours.

Embodiments of the dilator may include that the self-expanding dilator expands over a period of 0.5 to 2 hours.

Embodiments of the dilator may include that the driver includes an osmotically active agent.

Embodiments of the dilator may include that the driver includes an expandable polymeric matrix.

Embodiments of the dilator may include that the driver is configured to absorb water from the animal to expand the dilator.

Embodiments of the dilator may include a retaining anchor configured to retain the dilator in the Eustachian tube while the expandable portion expands from the non-expanded configuration to the expanded configuration.

Embodiments of the dilator may include that the dilator is configured to be removed from the Eustachian tube after the expandable portion expands from the non-expanded configuration to the expanded configuration.

Embodiments of the dilator may include a fluid passageway extending entirely through the dilator.

Embodiments of the dilator may include that the passageway is configured to equalize pressure within a middle ear of the animal with ambient pressure.

Embodiments of the dilator may include that the passageway is configured to drain liquid from a middle ear cavity of the animal.

Embodiments of the dilator may include that the dilator is sized and shaped to be inserted in, and dilate, a dysfunctional Eustachian tube of a human.

Embodiments of the dilator may include that the dilator has a flexural modulus of 4,300 psi or less.

In some embodiments, a kit that includes the subject dilator and instructions for the use thereof is provided.

Embodiments of the kit may include an insertion device configured to insert the dilator into the Eustachian tube of the animal.

### BRIEF DESCRIPTION OF THE FIGURES

FIG. 1 is a view of the human Eustachian tube, middle ear, and external auditory canal, with portions thereof shown in cross section, showing access to the

Eustachian tube from the nasal cavity and a Eustachian tube dilator mounted onto an insertion device according to embodiments of the present disclosure.

FIG. 2 is a view of the human Eustachian tube, middle ear, and external auditory canal, with portions thereof shown in cross section, in greater magnification and detail from that shown in FIG. 1, together with a Eustachian tube dilator mounted onto an insertion device.

FIG. 3 is a view of the human Eustachian tube, middle ear, and external auditory canal, with portions thereof shown in cross section, and a Eustachian tube dilator in an expanded configuration.

FIG. 4 is a side view of a Eustachian tube dilator in a non-expanded configuration according to embodiments of the present disclosure.

FIG. 5 is a perspective view of the dilator shown in FIG. 4 according to embodiments of the present disclosure.

FIG. 6 is an end view of the dilator shown in FIGS. 4 and 5 according to embodiments of the present disclosure.

FIG. 7 is a cross sectional view of the dilator shown in FIG. 6, along lines 7-7 according to embodiments of the present disclosure.

FIG. 8 is a side view of the dilator shown in FIGS. 4 through 7, mounted onto a dilator insertion device according to embodiments of the present disclosure.

FIG. 9 is an end view of the dilator and the dilator insertion device shown in FIG. 8 according to embodiments of the present disclosure.

FIG. 10 is a cross-sectional view of the dilator and the dilator insertion device shown in FIGS. 8 and 9 according to embodiments of the present disclosure.

FIG. 11 is a side view of the dilator shown in FIGS. 4 through 7, in an expanded configuration according to embodiments of the present disclosure.

FIG. 12 is a sectional view of the dilator shown in FIGS. 4 through 7, in an expanded configuration according to embodiments of the present disclosure.

FIG. 13 is a side view of another embodiment of a Eustachian tube dilator, in a non-expanded configuration according to embodiments of the present disclosure.

FIG. 14 is a cross-sectional view of a dilator shown in FIG. 13 according to embodiments of the present disclosure.

FIG. 15 is a side view of another embodiment of a Eustachian tube dilator in a non-expanded configuration according to embodiments of the present disclosure.

FIG. 16 is an end view of the dilator shown in FIG. 15 according to embodiments of the present disclosure.

FIG. 17 is a cross sectional view of the dilator shown in FIG. 15 immediately after the dilator has been inserted into the Eustachian tube according to embodiments of the present disclosure.

FIG. 18 is a cross sectional view of the device in FIG. 15, in an expanded configuration according to embodiments of the present disclosure.

FIGS. 19A-19C are sectional views of a dilator insertion device having a Eustachian tube dilator mounted thereon according to embodiments of the present disclosure.

FIG. 20 is a sectional view of another embodiment of a Eustachian tube dilator in a non-expanded configuration according to embodiments of the present disclosure.

FIG. 21 is a sectional view of the dilator shown in FIG. 20, in an expanded configuration according to embodiments of the present disclosure.

FIG. 22 is a sectional view of another embodiment of a Eustachian tube dilator in a non-expanded configuration according to embodiments of the present disclosure.

FIG. 23 is a sectional view of the dilator shown in FIG. 22, in an expanded configuration according to embodiments of the present disclosure.

FIG. 24 is a sectional view of another embodiment of a Eustachian tube dilator in a non-expanded configuration according to embodiments of the present disclosure.

FIG. 25 is a sectional view of another embodiment of a Eustachian tube dilator in a non-expanded configuration according to embodiments of the present disclosure.

FIG. 26 is sectional view of an osmotic engine and sleeve prior to assembly according to embodiments of the present disclosure.

FIG. 27 is a sectional view of another embodiment of a Eustachian tube dilator in a non-expanded configuration according to embodiments of the present disclosure.

Before the present invention is described in greater detail, it is to be understood that this invention is not limited to the particular embodiments described, and as such may, of course, vary. It is also to be understood that the terminology used herein is for the purpose of describing particular embodiments only, and is not intended to be limiting, since the scope of the present invention is embodied by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

It is noted that, as used herein and in the appended claims, the singular forms “a”, “an”, and “the” include plural referents unless the context clearly dictates otherwise. Thus, for example, reference to “a device” includes a single device as well as an assembly of devices, and reference to “a material” includes a single material as well as a combination of materials, and the like. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, it will be readily apparent to one of ordinary skill in the art in light of the teachings herein that certain changes and modifications may be made thereto without departing from the spirit and scope of the appended claims. Any recited method can be carried out in the order of events recited or in any other order which is logically possible.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. To the extent such publications may set out definitions of a term that conflict with the explicit or implicit definition of the present disclosure, the definition of the present disclosure controls. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

### DETAILED DESCRIPTION

The term “biocompatible” refers to the ability of a material or composition to be applied to tissues without eliciting significant inflammation, fibrosis, or tissue responses that are toxic, injurious or otherwise adverse.

The term “fluid” as used herein in its ordinary sense and refers to an at least partially gaseous and/or liquid substance that easily changes its shape. A fluid may contain a solid that is minimally, partially, or fully solvated, dispersed, or suspended. Examples of fluids include, without limitation, gases (such as oxygen, nitrogen, carbon dioxide, water vapor, and mixtures such as air), aqueous liquids (including water per se, salt water, and physiologic saline solutions), non-aqueous liquids (such as organic solvents, oils and the like), fluid emulsions, suspensions, and/or solutions such as mucus, blood, plasma, lymph, interstitial fluids, etc.

The term “fluid communication,” unless the context of its usage clearly indicates to the contrary, generally encompasses terms such as “air communication,” “liquid communication,” “bodily liquid communication” and “mucus communication.”

The terms “substantial” and “substantially” are referred to herein in their ordinary sense and are used to describe matters that are, e.g., considerable in importance, value, degree, amount, and/or extent. For example, a dilation device that is substantially anchored or substantially immobilized in the Eustachian tube is neither required nor precluded from absolute immobilization as long as movement of the device in the Eustachian tube is reduced to an inconsequential degree that does not compromise the intended functionality of the device within the Eustachian tube. Other uses of the term “substantially” involve an analogous definition.

The term “self-expanding” refers to dilators that expand simply by the action of placing the dilator at its intended site of action, e.g., within the Eustachian tube, and requires no external activation or other means to accomplish dilation of the dilator or the dilator's expansion driver. Thus, the term “self-expanding” excludes devices such as a balloon dilation catheter which are inflated using an external pump and accompanying inflation fluid conveying means.

The term “semipermeable” refers to membranes that are selectively permeable, being permeable to only certain molecules and not to all molecules or at least being much more permeable to certain molecules than to other molecules. In general, the term “semipermeable” refers to membranes that are permeable to water but have lower permeability to certain water soluble solutes (e.g., salts and osmopolymers) that are present in the devices and/or their environment of use.

The terms “osmosis”, “osmotic” and “osmotically” refer to the transmission of water molecules through a semipermeable membrane from an area of low solute concentration to an area of high solute concentration. Such transmission causes a pressure difference to develop across the membrane, which pressure difference acts as a driving force for at least some of the self-expanding drivers disclosed herein.

In general, the disclosures herein pertain to devices and methods for dilating a Eustachian tube of an animal, e.g., a human. The device (e.g., “dilation device” or “dilator”) is typically inserted in the Eustachian tube at a location closer to a nasopharynx than the tympanic membrane, e.g., adjacent to the torus tubarius orifice. The method of placement of the device is via the nasal or oral passageways and the through the nasopharyngeal opening of the Eustachian tube. As such, placement of the device does not require incision of the tympanic membrane, ear canal or entry into the middle ear.

Aspects of the present disclosure include a method of dilating a dysfunctional Eustachian tube in a patient. In certain embodiments, the method includes positioning a dilating device into the Eustachian tube. In some cases, the device includes an osmotic driver configured to expand an expandable portion from a non-expanded configuration to an expanded configuration, and the expandable portion disposed peripherally around the driver and configured to expand from the non-expanded configuration to the expanded configuration, where the non-expanded configuration is sized to be positioned within the Eustachian tube. In certain aspects, the dilator is positioned within the Eustachian tube in the region adjacent to the nasopharyngeal opening and thereby dilates that portion of the Eustachian tube. By “dilate” is meant that the average diameter of the Eustachian tube in the region in which the dilator is placed is greater after the device has expanded to its expanded configuration as compared to the average diameter of the Eustachian tube region before the device has expanded.

In some cases, the method further includes removing the device from the Eustachian tube. The device may be removed from the Eustachian tube at a point in time after insertion of the device into the Eustachian tube. For instance, the device may be removed from the Eustachian tube at a point in time after the device has expanded to the expanded configuration. The device may be removed by contacting a removal device to the device and extracting the device from the Eustachian tube. In some cases, the removal device may be attached to the device using a hook, a loop, a clamp, a suction device, a tether and the like. For instance, the removal device may include a hook configured to attach to a loop on the device. In certain embodiments, removal of the device is achieved by pulling the device from the Eustachian tube out through the nasopharyngeal opening. In certain embodiments, removal of the device may be facilitated by reducing the pressure exerted by the driver against the surrounding tissues before removing the device from the Eustachian tube. In some cases, the internal pressure of the driver may be reduced by puncturing the driver. For example, the removal device may include a needle or blade configured to create a hole in the driver allowing the internal pressure of the driver to equalize with the pressure in the nasopharynx. In some cases, the removal device may include a suction device configured to remove the internal contents of the driver from the device, thus reducing the pressure the device is exerting on the surrounding Eustachian tube.

In certain embodiments, the device can include a bioerodible or bioabsorbable material where the device removal occurs by bioerosion or bioabsorption of the device. The device may be left in the Eustachian tube of the subject and the device gradually erodes and may be absorbed by or expelled by the patient's body over a period of time.

Aspects of the present disclosure include inserting the device through a nostril or mouth of a patient into a dysfunctional Eustachian tube of the patient. After insertion, the device is left in place in the Eustachian tube for an extended period of time during which the size of the device slowly expands exerting pressure on the Eustachian tube to gradually dilate the Eustachian tube. In order to minimize patient discomfort, the expansion of the device may occur over a period of 0.5 hour or more, 1 hour or more, 2 hours or more, 3 hours or more, 4 hours or more, 5 hours or more, 6 hours or more, 7 hours or more, or 8 hours or more, 12 hours or more, 24 hours or more, 48 hours or more, 72 hours or more, etc.

In certain embodiments, the method includes contacting the device with a fluid prior to positioning the device in the dysfunctional Eustachian tube. Contacting the device with a fluid prior to positioning the device in the Eustachian tube may initiate expansion of the device and/or reduce the time required to initiate expansion of the device in the Eustachian tube. For embodiments of the device that include a swellable polymer or an osmotic agent, contacting the device with a fluid prior to insertion into the Eustachian tube may facilitate expansion of the device after positioning the device in the tube. For example, embodiments of the device may be configured to begin expanding 30 min or more, such as 45 min or more, including 60 min or more, or 90 min or more, 120 min or more, or 180 min or more after the device has been contacted with a fluid. In these embodiments, contacting the device with a fluid prior to insertion of the device into the Eustachian tube may facilitate the onset of expansion of the device at a point in time sooner after insertion of the device into the Eustachian tube. In some instances, the fluid may include water, saline, sterile water, sterile saline, and the like.

In certain embodiments, the method includes delivering a drug from the device while the device is positioned within the Eustachian tube. For example, the drug may include, but is not limited to, an antibiotic, an anti-inflammatory drug, anesthetics (e.g., local anesthetics), analgesics (e.g., locally acting analgesics), vasoconstrictors, combinations thereof, and the like, as discussed above. The drug may be delivered to the tissues of the Eustachian tube that surround the device when the device is positioned within the Eustachian tube. In some cases, the drug may be delivered to the tissues at the end of the Eustachian tube adjacent to the nasopharyngeal opening or into the nasopharynx.

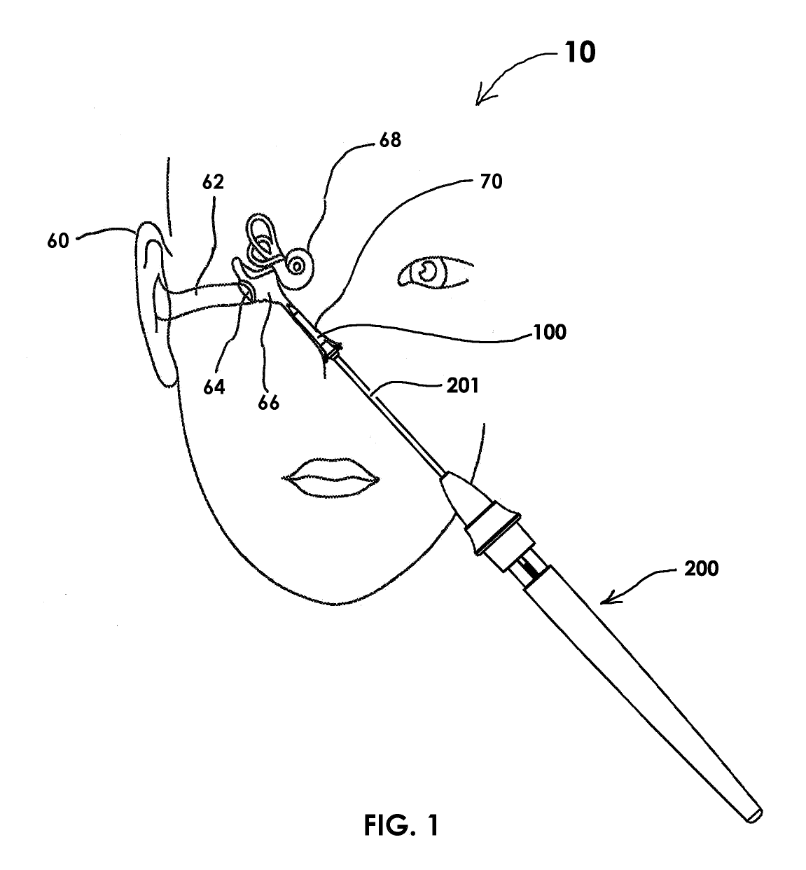

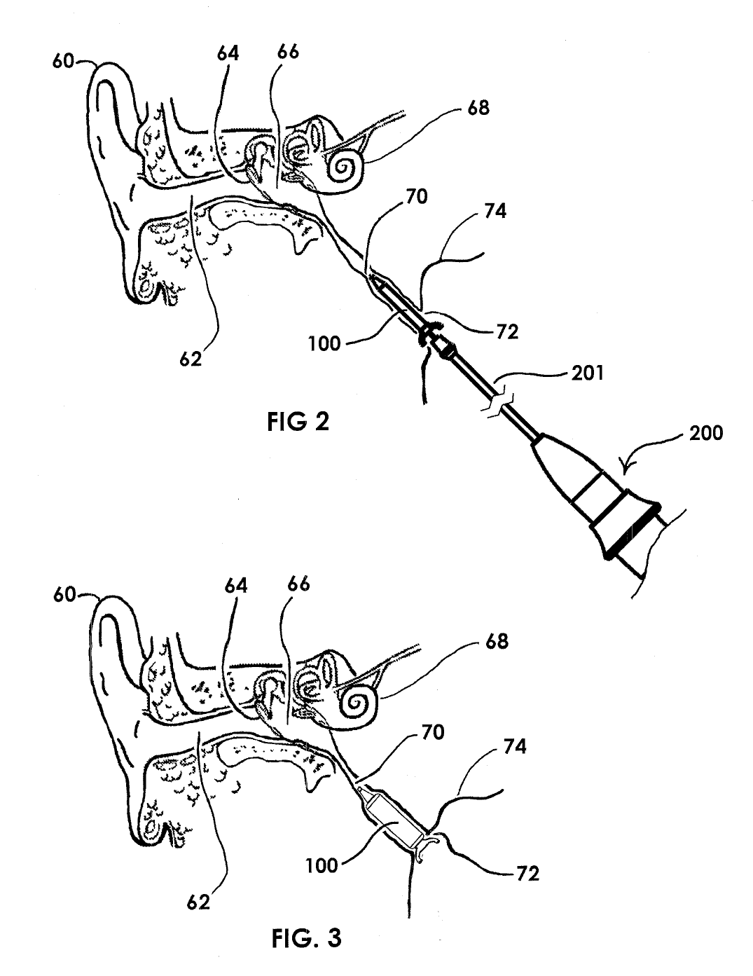

Referring now to FIGS. 1 and 2, there is shown a subject 10 having an ear 60 with an external auditory canal 62, a tympanic membrane 64, a middle ear cavity 66, a cochlea 68 and a Eustachian tube 70 having a nasopharyngeal orifice 72 providing access from the nasopharynx 74. An insertion device 200 having a cannula 201 with a dilator 100 mounted on the distal end thereof shows how the dilator 100 can be inserted into Eustachian tube 70 through the subject's nostril and nasal cavity. Alternatively (not show in the figures) the nasopharyngeal orifice 72 of the Eustachian tube 70 can be accessed via the subject's mouth.

FIG. 3 shows the dilator 100 in an expanded configuration. As can be seen, the dilator sits in the portion of the Eustachian tube which is closer to the nasopharynx 74 than the portion of the Eustachian tube that is closer to the middle ear cavity 66. This portion of the Eustachian tube tends to be soft tissue, not bone. As is explained in more detail hereinafter, the dilator 100 is self-expanding. As such, the dilator requires no external manipulation or intervention on the part of the physician to start the expansion other than to simply place the device in the Eustachian tube. In the case of dilator 100 which is osmotically driven, water which is naturally present in the tissues of and adjacent the Eustachian tube is taken up by dilator 100 and causes it to expand. In certain instances, the dilator 100 has an unexpanded diameter of 1 to 5 mm, such as 1 to 4 mm, or 1 to 3 mm, or 2 to 3 mm, and is designed to expand to an expanded diameter of 2 to 10 mm, such as 2 to 9 mm, or 2 to 8 mm, or 3 to 8 mm, or 4 to 8 mm, or 4 to 7 mm or 4 to 6 mm. In some cases, the dilator 100 has an unexpanded diameter of 2 to 3 mm, and is designed to expand to an expanded diameter of 4 to 8 mm.

The dilator 100 can be used to equalize the pressure in the middle ear cavity 66 with ambient (e.g., atmospheric) pressure. In such applications, the dilator is designed to expand from its unexpanded configuration to its expanded configuration in less than 4 hours, and in certain embodiments less than 2 hours. The expansion desirably takes place over a period of 0.5 hours or more to avoid sudden impact and damage to the Eustachian tube and to avoid patient discomfort and the need for topical anesthetics and/or patient anesthesia. Upon expansion, the dysfunctional Eustachian tube eventually opens and gas is allowed to pass through a passageway within the dilator 100 in order to equalize the pressure in the middle ear cavity 66 with ambient pressure. The dilator 100 can thereafter be removed from the Eustachian tube 70.

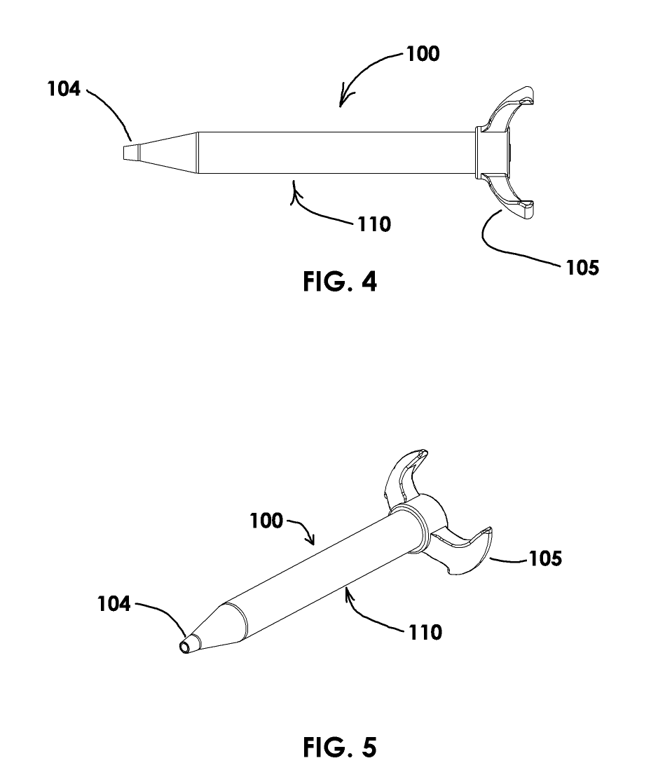

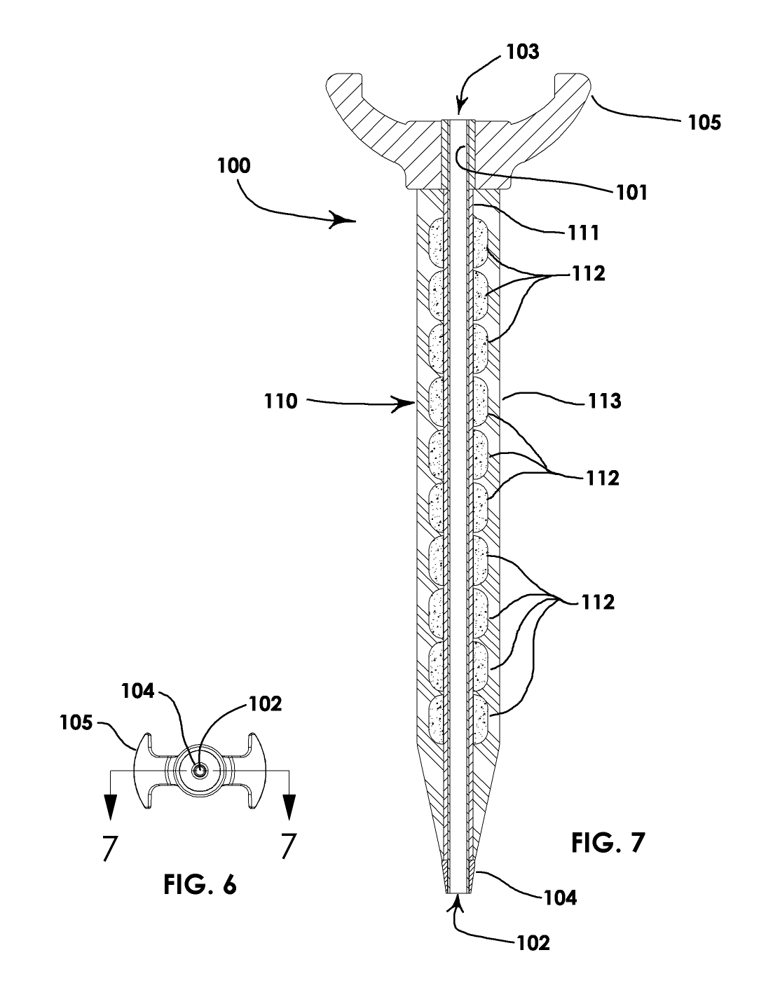

The Eustachian tube dilator 100 is shown in greater detail in FIGS. 4 through 7. FIGS. 4 and 5 show the dilator 100 in a non-expanded configuration which is the configuration at the time the device is positioned within a nasopharyngeal opening of the Eustachian tube 70. Dilator 100 has a tapered distal tip 104, a proximal anchor 105 configured to prevent the dilator from being inserted too far into the Eustachian tube 70, and an osmotic driver 110. As shown in FIGS. 6 and 7, dilator 100 includes a conduit 101 having a distal opening 102 and a proximal opening 103. As is explained in more detail herein, conduit 101 has an inner diameter of 0.1 to 1 mm or more in order to permit gas to pass into and/or out of the middle ear cavity 66 while the device 100 is positioned within, and has expanded, the Eustachian tube 70. For example, the conduit 101 may have an inner diameter of 0.1 to 2 mm, such as 0.2 to 2 mm, including 0.2 to 1.5 mm, or 0.2 to 1 mm, or 0.3 to 1 mm, or 0.4 to 1 mm, or 0.5 to 1 mm. The conduit 101 is non-collapsible under the pressures exerted by the osmotic driver 110 during use, so that as osmotic pressure is generated within driver 110, it causes the device to expand outward from the conduit 101 rather than causing the conduit 101 to collapse or significantly decrease in diameter. The conduit 101 can be relatively rigid or bendable along its length.

Positioned at the distal opening 102 is a tapered tip 104. As used herein, the term “distal” refers to the end of the device that is inserted through the nasopharyngeal orifice 72 of the subject and remains within the Eustachian tube 70 during use. Similarly, the term “proximal” refers to the end of the dilator that remains in or near the nasopharyngeal orifice 72 when the dilator 100 is positioned in the Eustachian tube 70 during use.

At the proximal end of dilator 100 is a proximal anchor 105 having a pair of radially extending members, which as shown in FIG. 6, extend in opposite directions from the central axis of dilator 100. Proximal anchor 105 has a greater diameter compared to the unexpanded diameter of osmotic driver 110 and thereby prevents the dilator 100 from being inserted too far into the Eustachian tube70 thereby preventing the tip 104 from impacting and/or damaging the tympanic membrane 64. The hollow interior of conduit 101 creates a conduit or passageway for fluid and/or gas, such as air, mucus, puss and/or blood, to pass through the dilator 100 while the dilator 100 is positioned within the Eustachian tube 70.

Positioned along a central portion of conduit 101 (e.g., between the distal tip 104 and the proximal anchor 105 is an osmotic driver 110 that includes an inner membrane 111 and an outer membrane 113 that together surround an osmotic core comprised of a plurality of osmotic tablets 112. The osmotic tablets 112 may include one or more osmotically active agents such as water soluble salts or sugars, such as sodium chloride, lactose, etc., and optionally binders, lubricants and mold release agents. The osmotic core additionally may include osmopolymers such as polyethylene oxide, sodium carboxymethyl cellulose, and the like. Certain embodiments of the osmotic core include ring or torus (e.g., donut) shaped salt- and polymer-containing tablets having an inner opening that is large enough to slide over conduit 101 with inner membrane 111. In some instances, the tablets 112 have an outer diameter of 5 mm or less, such as 4 mm or less, or 3 mm or less, or 2 mm or less. For instance, the tablets 112 may have an outer diameter of 3 mm. In some instances, the tablets are composed of an osmotically active salt (e.g., NaCl) and/or polymer, such as a high molecular weight hydrogel-forming polymer, for example polyethylene oxide (e.g., Polyox™, Dow Chemical Company, Midland, Mich.). In certain cases, the tablets 112 include tableting excipients and/or lubricants. In some embodiments, the tablets 112 include 10 to 95 wt % salt, such as 20 to 90 wt % salt, including 30 to 80 wt % salt, or 40 to 70 wt % salt. For example, the tablets 112 may include 10 to 95 wt % NaCl, such as 20 to 90 wt % NaCl, including 30 to 80 wt % NaCl, or 40 to 70 wt % NaCl. In some cases, the tablets 112 include 30 to 80 wt % NaCl. In certain embodiments, the tablets 112 include 5 to 90 wt % polymer, such as 10 to 80 wt % polymer, including 20 to 70 wt % polymer, or 30 to 60 wt % polymer. For example, the tablets 112 may include 5 to 90 wt % Polyox, such as 10 to 80 wt % Polyox, including 20 to 70 wt % Polyox, or 30 to 60 wt % Polyox. In certain cases, the tablets 112 include 20 to 70 wt % Polyox. In some embodiments, the tablets 112 are composed of a salt and a polymer, as described above. For example, the tablets 112 may include 30 to 80 wt % NaCl and 20 to 70 wt % Polyox. In certain instances, the NaCl gives a faster rate of expansion than does the Polyox, though both materials are osmotically active and cause water to be imbibed into the interior of the osmotic driver 110. Because of its low molecular weight, there may be some leakage of NaCl out through the semipermeable membrane 113, whereas because of its high molecular weight, there is substantially no leakage of the Polyox out through the semipermeable membrane 113. A higher NaCl loading (e.g., 80 wt %) gives a shorter duration of dilator expansion than a lower NaCl loading (e.g., 20 wt %).

Once dilator 100 is inserted into a Eustachian tube 70, water from the patient's body permeates through the membrane 113 by osmosis and forms a solution of the salt or sugar and hydrates the osmopolymer in the osmotic tablets 112, thereby causing the osmotic tablets 112 to expand. As water imbibes in, the volume of the tablets 112 increases. In addition, due to its elastic nature, the membrane 113 also expands to accommodate the increased volume of the osmotic tablets 112. The rate of water permeation can be controlled by controlling the composition, thickness and porosity of the membrane 113, in combination with the osmotic activity of the tablets 112. In certain embodiments of the devices disclosed herein, the membrane 113 composition, thickness and porosity are controlled to achieve expansion of the tablets 112 over a period of 0.5 hours or more, such as 1 hour or more, including 2 hours or more, or 3 hours or more, or 4 hours or more. In other embodiments of the devices disclosed herein, the membrane 113 composition, thickness and porosity are controlled to achieve expansion of the tablets 112 over a period of 4 hours or more. In this way, the rapid expansion and the resulting pain experienced by the patient during balloon catheter procedures may be substantially avoided.

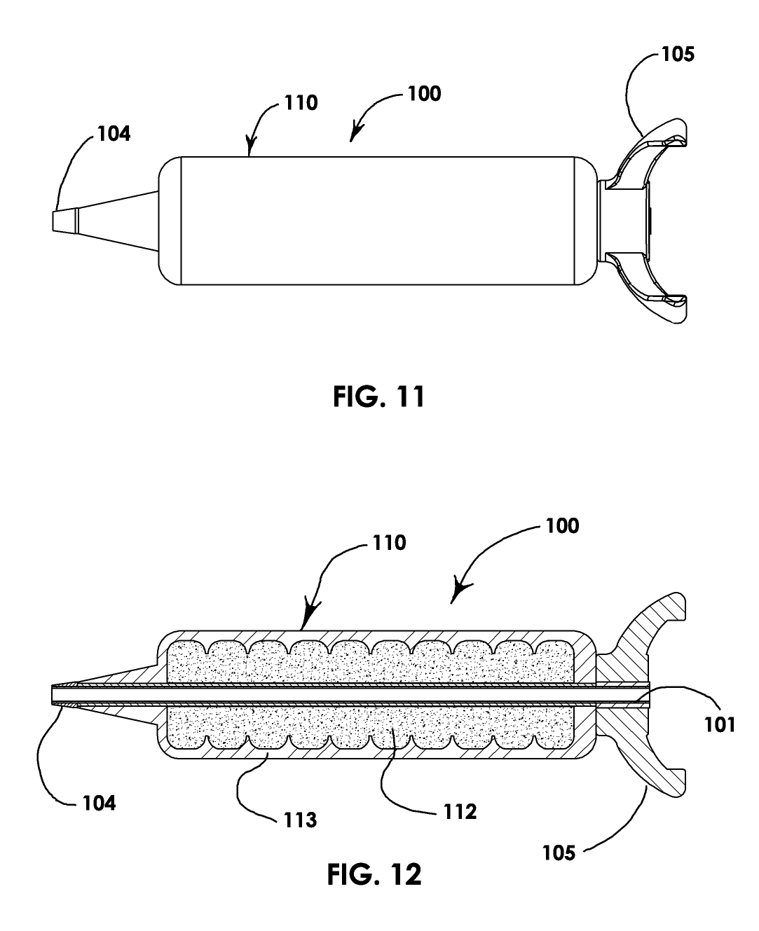

Referring now to FIGS. 11 and 12, there is shown the dilator 100 in an expanded configuration after it has been in place within a Eustachian tube. The sectional view of FIG. 12 relates to FIG. 11 in the same way as the sectional view of FIG. 7 relates to FIG. 4. As can be seen by a comparison of FIGS. 7 and 12, the volume of the osmotic tablets 112 has expanded due to the imbibed water and the expansion of the elastic semipermeable membrane 113 has expanded to accommodate this increased volume. In this way, the diameter of the tablets 112 has increased and when in place within the Eustachian tube exerts an outward radially expansive force thereon, causing the adjacent portions of the Eustachian tube to dilate.

Optionally, the dilator 100 may be configured to release a drug while in place within the Eustachian tube 70. The dilator may be preloaded with a drug. For example a drug can be loaded into the tablets 112. In certain instances, the drug is water soluble and thereby acts itself as an osmotic agent. In some cases, the drug is present in a coated layer upon the surface of the dilator. The coated layer may include a blend of film former polymer mixed with the drug. In yet another embodiment, the drug is blended directly into membrane 113 which drug elutes in situ directly to tissues lining the Eustachian tube. Alternatively, a drug may be coated onto the outer surfaces of dilator 100 by the physician immediately before use. For example, the dilator 100 can be sprayed, dipped or coated with a drug solution or gel formulation that includes a drug prior to placement of dilator 100 within the patient. The drug added to dilator 100 may be selected from antibiotics, anti-inflammatory drugs, anesthetics (e.g., local anesthetics), analgesics (e.g., locally acting analgesics), drugs that reduce bleeding (e.g., vasoconstrictors), combinations thereof, and the like. In certain embodiments, antibiotics include levofloxacin, moxifloxacin, amoxicillin, clavulanic acid, clarithromycin, azithromycin, cefuroxime, ciprofloxacin, salts thereof and combinations thereof and the like. In some instances, anti-inflammatory drugs include budesonide, mometasone, prednisone, methylprednisolone, dexamethasone, salts thereof and combinations thereof and the like. In some cases, local anesthetics include lidocaine, bupivacaine, ropivacaine, tetracaine, salts thereof and combinations thereof and the like. In certain embodiments, locally acting analgesics include: acetaminophen; Cox-2 inhibitors, such as celecoxib and rofecoxib and the like; NSAIDS such as diclofenac, ibuprofen, ketoprofen, naproxen, piroxicam, aspirin and the like; opioids such as morphine; opioid agonists such as tramadol and the like. In certain embodiments, vasoconstrictors include oxymetazoline, epinephrine, tranexamic acid, salts thereof, combinations thereof, and the like. In certain instances, the drug reservoirs may include a combination of drugs, such as a combination of an NSAID, an anti-inflammatory drug and a vasoconstrictor. For example, the drug may include OMS103HP (Omeros Corp., Seattle, Wash.), which includes an NSAID (ketoprofen), an anti-inflammatory drug (amitriptyline) and a vasoconstrictor (oxymetazoline).

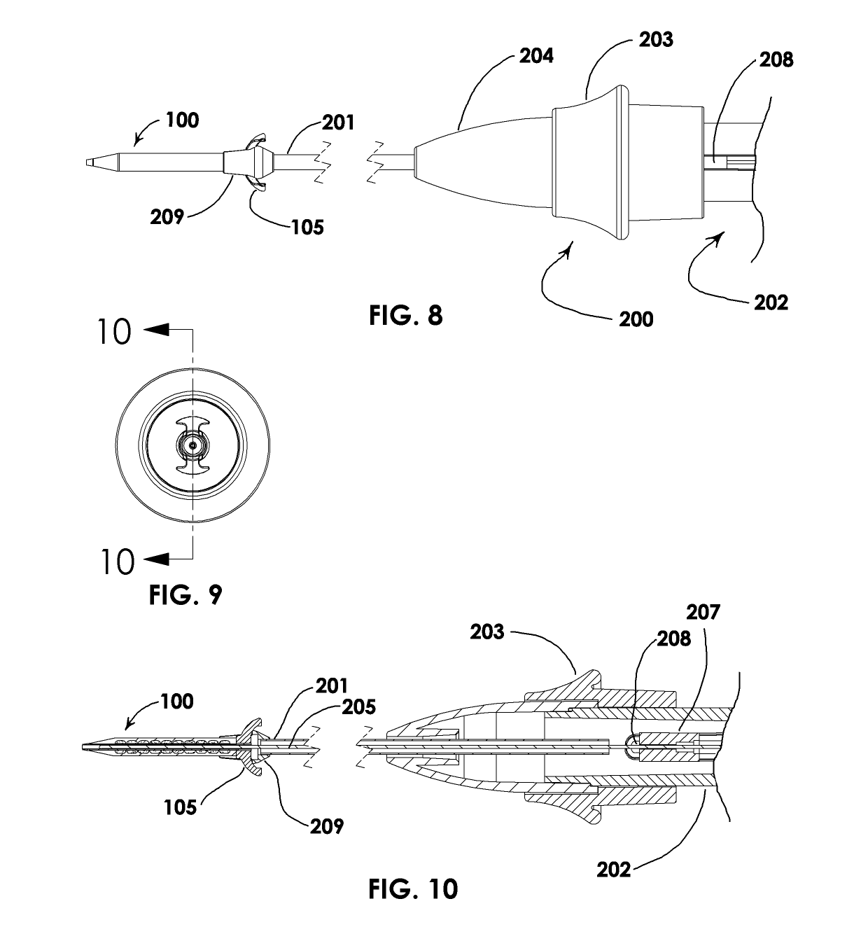

One embodiment of a Eustachian tube dilator insertion device 200 is shown in FIGS. 8 to 10. In some instances, the insertion device facilitates insertion of the dilator into the Eustachian tube of the patient. Insertion device 200 has a handle 202, a cannula 201 mounted on the distal end 204 of handle 202, the handle 202 having a slidable trigger 203 which is connected to member 207 via slot 208. A rod 205 is slidably positioned within cannula 201. The rod 205 can be for example made from metal or plastic and has a diameter just slightly less that the inner diameter of dilator conduit 101. The proximal end of rod 205 is attached to member 207 by conventional means. With the sliding trigger 203 oriented in the distal-most position (e.g., the left-most position as shown in FIGS. 8 and 10), the dilator 100 is mounted on the distal end of insertion device 200 and may be deployed into a Eustachian tube 70. In this position, the rod 205 extends out of the distal end of cannula 201 and extends into the lumen of conduit 101 of dilator 100. In certain embodiments, the rod 205 occupies substantially the entire lumen of conduit 101 which may facilitate a minimization of clogging of the distal opening 102 by body fluids or cellular matter during insertion of the dilator 100 into a Eustachian tube 70. As shown in FIGS. 8 and 10, the distal end of cannula 201 has a slotted flange 209. The radially extending portions of proximal anchor 105 extend out of the slots of flange 209 and may minimize axial rotation of the dilator 100 during insertion into a Eustachian tube 70.

In use, the mounted dilator 100, and cannula 201 are advanced either through the subject's nostril or mouth, into the nasopharynx 74 and then through the nasopharyngeal orifice 72 until the proximal anchor 105 abuts against the tissue surrounding the nasopharyngeal orifice 72. Because the proximal anchor abuts against the ends of the slots in flange 209, the dilator can be pushed into a narrowed, stenotic and/or completely closed Eustachian tube 70 by the physician applying a distally oriented pushing force via the handle 202. Once in position within the Eustachian tube 70, the physician slides the trigger 203 to the proximal position (e.g., the right-most position as shown in FIGS. 8 and 10), and the rod 205 is withdrawn from the interior lumen of conduit 101, thereby releasing the dilator in the Eustachian tube. The insertion device 200 may then be withdrawn.

In certain embodiments, device 200 includes a light source (not shown in the figures), which in some instances is a directional light source, such as a low energy laser. The light source emits light into the lumen of cannula 201 using known light directing means and a light-reflecting interior surface of cannula 201. In some embodiments, rod 205 and dilator 100 are also constructed of light transmitting and/or translucent materials so that the light from the light source causes at least portions of the dilator 100 to become illuminated. The illumination may have sufficient intensity so that the emitted light can be seen through the patient's facial tissue. The position of the illuminated dilator 100 may help the physician to correctly position the dilator in the Eustachian tube. Alternatively, the dilator 100 described herein may be placed using an illuminated guide wire that extends through the cannula 201 and/or through rod 205 and optionally through the internal lumen of conduit 101.

Other suitable dilator insertion devices are disclosed in FIGS. 3-12, 18-20 and 24 of U.S. patent application Ser. No. 13/219,497 filed Aug. 26, 2011, and also in U.S. Provisional Patent Application, titled “Devices and Methods for Dilating a Paranasal Sinus Opening and for Treating Sinusitis,” filed concurrently with the present application, the disclosures of each of which are incorporated herein by reference in their entirety.

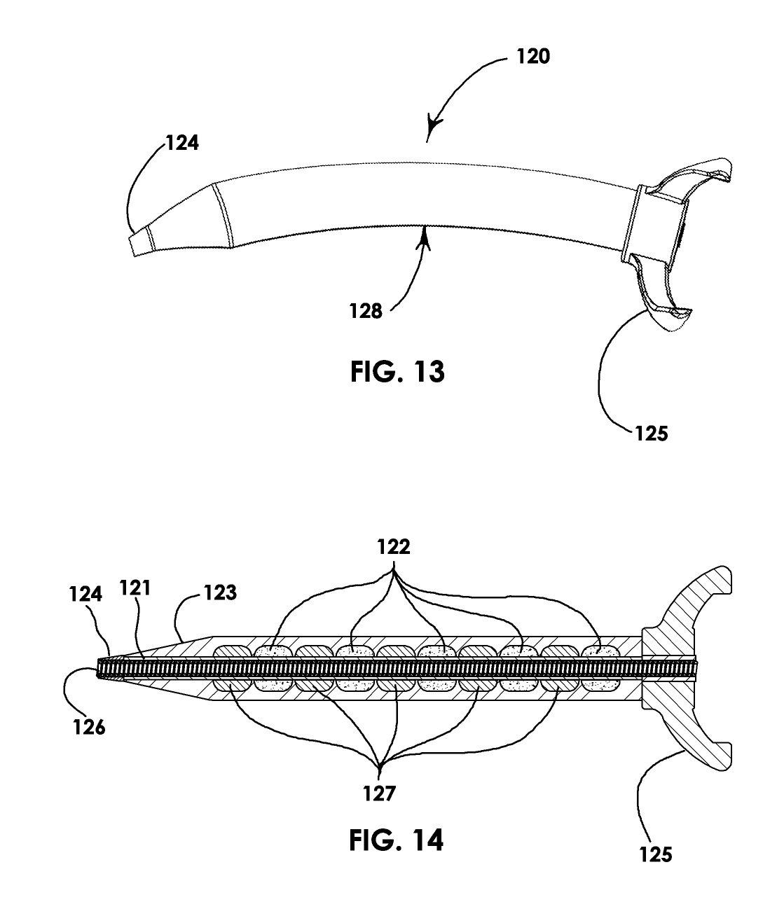

Referring now to FIGS. 13 and 14, there is shown another embodiment of a Eustachian tube dilator 120 having increased flexibility which is useful in those cases where the subject's Eustachian tube 70 has more substantial bends along its length either due to disease or genetic causes. In certain embodiments, dilator 120 has a flexural modulus of 500 to 4300 psi. For example, the dilator may have a flexural modulus of 500 to 5000 psi, such as 750 to 4500 psi, including 1000 to 4500 psi, or 1500 to 4000 psi, or 2000 to 3500 psi, or 2500 to 3000 psi. In other embodiments, dilator 120 has a flexural modulus of 2900 psi. Dilator 120 also has a tapered distal tip 124, a proximal anchor 125, an osmotic driver 128 comprised of an inner membrane 121, an outer elastic semipermeable membrane 123, and osmotic tablets 122, all of which perform similar functions as their counterparts in dilator 100. Unlike dilator 100, dilator 120 has an increased flexibility and bendability, as shown in FIG. 13. The elements of dilator 120 which increase bendability are shown in FIG. 14 and include a coiled spring 126 in place of conduit 101 and annular-shaped elastic spacers 127 positioned between the tablets 122. Spring 126 resists the radial compression forces encountered during the expansion of the tablets 122 so that the interior lumen of the spring 126 remains open during expansion of dilator 120. The spring can be tightly coiled such that adjacent turns are touching one another in order to provide axial rigidity in those cases where the physician needs to apply greater force to insert the dilator 120 into the Eustachian tube 70. The spacers 127 can be made from known elastic materials, such as polymers and rubbers that are easily compressed and/or flexed. In those cases where the dilator 120 is to be inserted for relatively short periods of time (e.g., 4 hours or less), the spacers may be made from materials that exhibit low water absorption so that any water taken up by the osmotic driver 128 is directed to radial expansion of the dilator 120 and not to saturation of the spacers 127. Suitable materials for spacers 127 include closed cell polyethylene foams and neoprene rubber.

An alternate configuration of a Eustachian tube dilation device (not shown in the figures), but which is similar to dilator 100 shown in FIGS. 4, 5, 11 and 12, has a rigid or non-collapsible tubular semipermeable membrane in place of impermeable conduit 101 and an elastic impermeable membrane in place of elastic semipermeable membrane 113. Such a device expands by absorption of water vapor present in the lumen of the tubular semipermeable membrane. In certain embodiments, under similar conditions, osmotic engine water absorption from 100% relative humidity water vapor is about two orders of magnitude lower than when the same osmotic engine is in contact with bulk water. The use of an “interior” semipermeable osmotic membrane, as described above, may be adapted for applications where the dilation device expansion takes place over a longer period of time. For applications where dilation device expansion takes place over a shorter period of time (e.g., 0.5 to 4 hours), an internal semipermeable membrane dilation device may utilize a water wicking element, for example a hydrophilic fabric or similar material, within the interior lumen of the tubular membrane and optionally extending out past the proximal and/or distal ends of the dilation device.

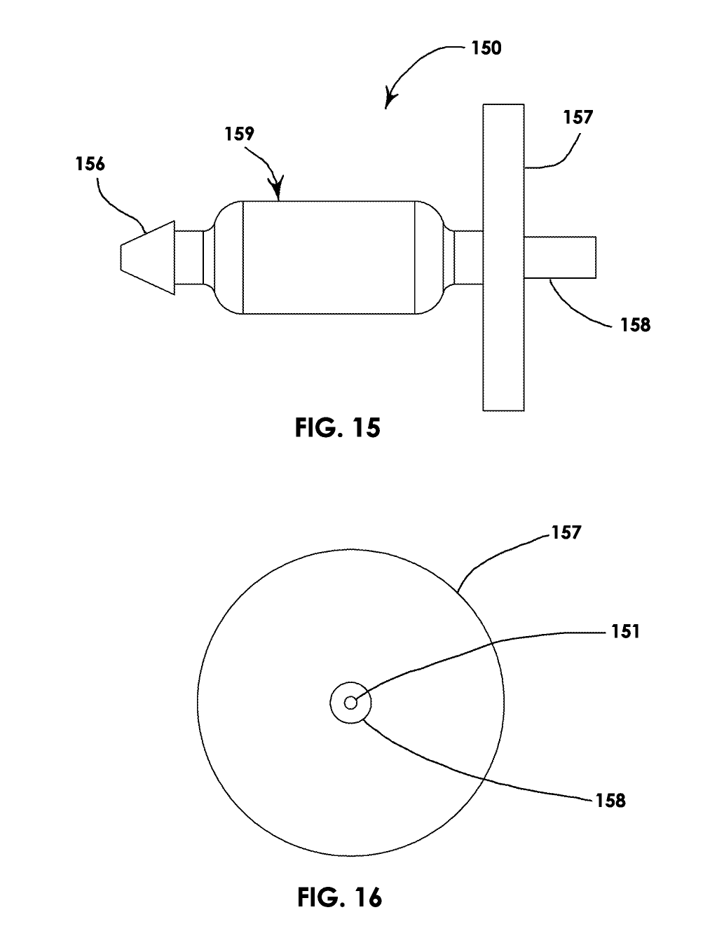

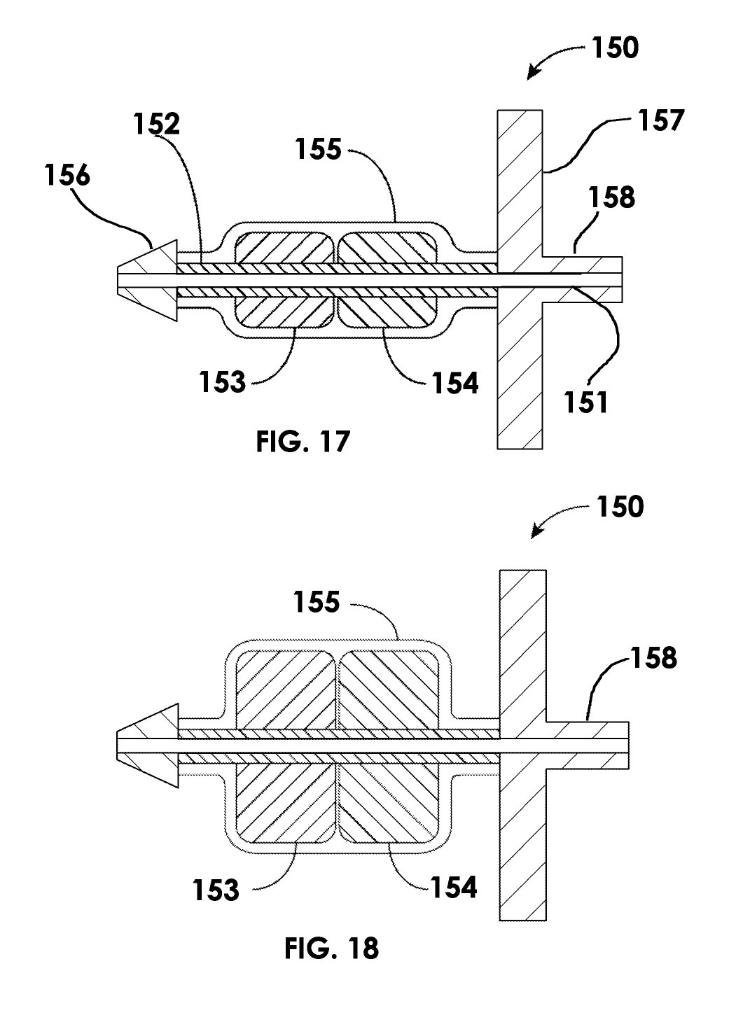

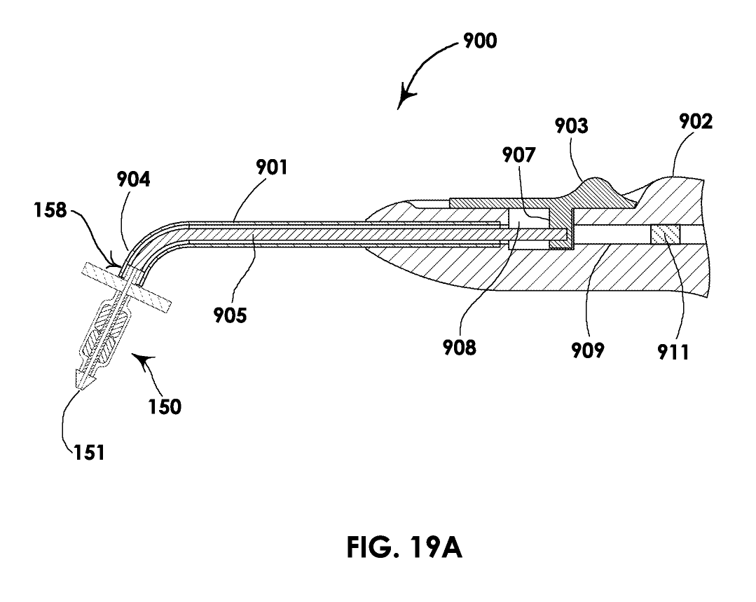

Another embodiment of an osmotic dilator 150 is shown in a side view in FIG. 15 and in an end view in FIG. 16. The dilator 150 includes an osmotic driver 159, a tapered distal tip 156, a proximal anchor 157 and a mounting member 158. Dilator 150 is shown in greater detail in cross sectional views of FIGS. 17 and 18. Dilator 150 is shown in a non-expanded configuration in FIG. 17 and in an expanded configuration in FIG. 18. Dilator 150 includes tube 151 (e.g., a bendable plastic tube which will not collapse under the pressures exerted by the osmotic driver 159) having the osmotic driver 159 disposed thereon. The driver 159 is comprised of an inner membrane coating 152 disposed on the tube 151, two osmotic tablets 153, 154 threaded onto the membrane-covered tube 151 and an external elastic semipermeable membrane coating 155 applied thereover. The dilator 150 includes an optional tapered distal tip 156 that can be formed of plastic, metal or ceramic which may be secured to the tube 151, e.g., by gluing and which makes it easier for the medical practitioner to insert the dilator into the nasopharyngeal orifice 72. The proximal anchor 157 may also be secured to the tube 151. For example, by gluing and/or by screw threads provided on the exterior surface of the distal end of tube 151 and matching threads on the interior of tip 156. The tapered shape of distal tip 156 facilitates insertion of the dilator into the nasopharyngeal orifice 72 by the physician. The proximal anchor 157 and mounting member 158 may also be secured (e.g., by gluing and/or screw threads) to the tube 151. The tube 151 is open at both the proximal and distal ends of dilator 150, and thereby allows gas to pass through the dilator which aids in the equalization of pressure in the middle ear cavity 66 with ambient air pressure. Similarly, tube 151 permits bodily fluid to drain out of the middle ear cavity while the dilator 150 is positioned in the Eustachian tube.

As described herein, the proximal anchor 157 has a sufficient diameter, e.g., 8 to 12 mm, and stiffness that it cannot be easily pushed through the nasopharyngeal orifice 72 during dilator 150 placement.

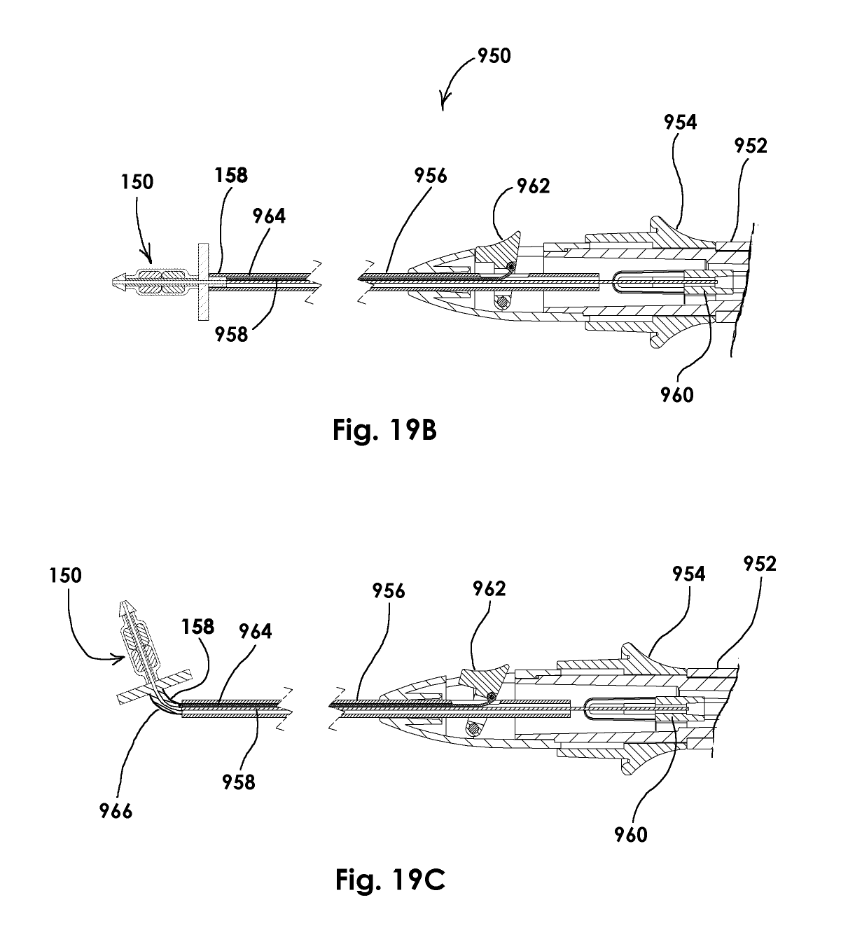

Another embodiment of a Eustachian tube dilator insertion device 900 is shown in FIG. 19. Device 900 has a handle 902 with a hollow internal lumen 909, an hollow elongated member 901 mounted on the handle 902 within lumen 909, the member 901 having a curved distal tip section 904 and a slidable trigger 903 with trigger arm 907 which moves back and forth within slot 908.

In certain embodiments, the distal tip of the insertion device is configured to flex from 0 to 120 degrees, such as 0 to 110 degrees, including 0 to 100 degrees, or 0 to 90 degrees, or 0 to 80 degrees, or 0 to 70 degrees, or 0 to 60 degrees relative to the longitudinal axis of the insertion device. In some cases, the distal tip of the insertion device is configured to flex from 0 to 80 degrees relative to the longitudinal axis of the insertion device. In certain instances, the distal tip of the insertion device is sufficiently flexible to bend to 80 degrees at a radius of 3 to 12 mm.

A flexible rod 905 is slidably positioned within member 901. The rod 905 can be for example made from flexible polymers having a diameter slightly less than the diameter of lumen 909. The rod 905 may be sufficiently flexible to bend as it is advanced through the curved tip section 904 of hollow elongated member 901. The proximal end of rod 905 is attached to trigger arm 907 by conventional means. With the trigger 903 in the non-advanced position (the right-most position as shown in FIG. 19), the distal end of rod 905 is recessed within the curved tip section 904, thereby allowing sufficient space to mount osmotic dilator 150 thereon by inserting mounting member 158 into the distal tip of hollow member 901. By moving the trigger to the advanced position (e.g., the left-most position as shown in FIG. 19), the dilator 150 is pushed off the distal end of member 901. Thus in use, the insertion device 900 with dilator 150 mounted thereon is advanced through the patient's nasal or oral cavity into the nasopharynx and the distal end of dilator 150 is then pushed into the nasopharyngeal orifice 72 of Eustachian tube 70. Once in place within the Eustachian tube, the trigger 903 is advanced which releases dilator 150 from the insertion device 900 and the insertion device is then withdrawn.

In certain embodiments, device 900 includes a visible light source 911, which in some instances is a directional light source, such as a low energy laser. The light source 911 emits visible light into the lumen of hollow member 901. When the light source 911 is positioned as shown in FIG. 19, the trigger arm 907 may be off set with respect to the position of light source 911 to allow the light to reach the lumen of member 901, or the arm 907 may be constructed of a light-transmitting material such as clear plastic or glass. In some embodiments, rod 905 and dilator 150 are also constructed of light transmitting and/or translucent materials so that the light from the light source 911 causes at least portions of the dilator 150 to become illuminated. The illumination may have sufficient intensity so that the emitted light can be seen through the patient's facial tissue. The position of the illuminated dilator 150 may help the physician to correctly position the dilator in the Eustachian tube. As an alternative to the light source 911, the osmotic dilator 150 described herein may be placed using an illuminated guide wire that extends through the rod 905 and optionally through the internal lumen of the osmotic dilator 150.

Another embodiment of a Eustachian tube dilator insertion device 950 is shown in FIGS. 19B and 19C. Device 950 has a handle 952 with a hollow elongated member 956, a flexible multi-lumen section 966 mounted on the handle 952 within hollow elongated member 956, the flexible multi-lumen section 966 includes a center lumen and at least one lumen that is positioned off-axis. A cable 964 is affixed to the distal end of the off-axis lumen (e.g., the end of the off-axis lumen that is furthest away from the handle) and runs the entire length of flexible multi-lumen section 966 and attaches to a deflection trigger 962 by conventional means. The backward sliding movement of the trigger 962 (e.g., movement of the deflection trigger in a proximal direction) applies tension to the cable 964 causing the distal tip of flexible multi-lumen section 966 to flex and bend. In some embodiments, the tip of flexible multi-lumen section 966 has a range of motion of 0 to 90 degrees relative to the longitudinal axis of the insertion device. When the deflection trigger 962 is moved forward (e.g., movement of the deflection trigger in a distal direction), tension on the cable 964 is released and the tip of flexible multi-lumen section 966 straightens. In the central lumen of flexible multi-lumen section 966, a flexible rod 958 is slidably positioned. The rod 958 may be sufficiently flexible to bend as it is advanced through the curved tip section of the flexible multi-lumen section 966. The proximal end of rod 958 is attached to trigger arm 960 by conventional means. With the trigger 954 in the non-advanced position (e.g., the right-most position as shown in FIGS. 19B and 19C), the distal end of rod 958 is recessed within the center lumen of flexible multi-lumen section 966, thereby allowing sufficient space to mount osmotic dilator 150 thereon by inserting mounting member 158 into the distal end of flexible multi-lumen section 966. By moving the trigger to the advanced position (e.g., the left-most position as shown in FIGS. 19B and 19C), the dilator 150 is pushed off the distal end of flexible multi-lumen section 966 and into the nasopharyngeal orifice 72 of Eustachian tube 70.

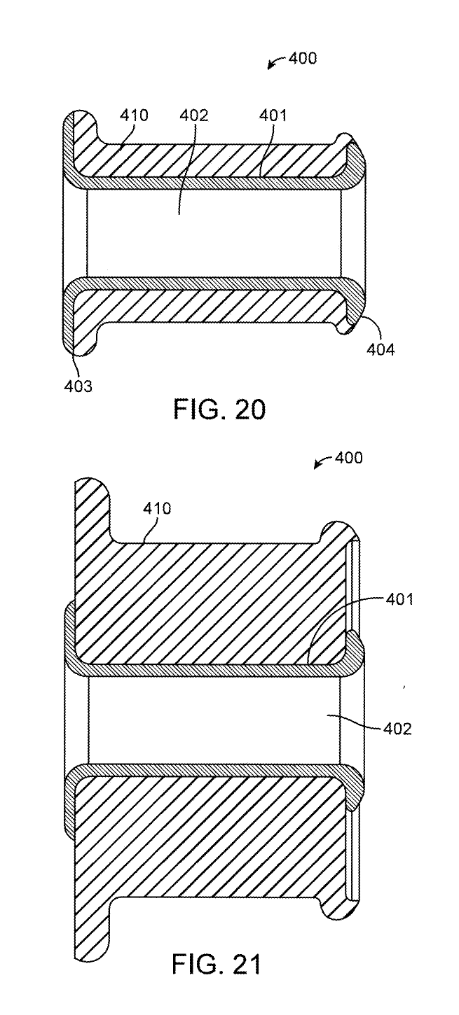

Referring now to FIGS. 20 and 21, there is shown an embodiment of a Eustachian tube dilation device 400 with an expandable driver including a polymer matrix. Similar to devices 100 and 150, device 400 also has an inner conduit 401 composed of a substantially rigid or non-collapsible polymer, ceramic, metal, composite, and the like. The interior of conduit 401 is open, creating a passageway 402 for allowing bodily fluids such as mucus, puss and blood to drain out of the middle ear cavity 66 and air to pass into and out of the cavity 66 while the dilation device 400 is positioned within the nasopharyngeal orifice 72 and Eustachian tube 70. The proximal end of conduit 401 is flared, creating an anchoring flange 403 which helps keep device 400 from being inserted beyond opening 72 and into Eustachian tube 70 during placement and from migrating further into Eustachian tube 70 during use.

Surrounding conduit 401 is an expandable driver 410 that includes an expandable polymer matrix. Suitable expandable polymers for use as the driver 410 include water swellable polymers such as polyethylene oxide (PEO), hydroxypropylmethyl cellulose, polyvinyl alcohol, carboxymethylcellulose, sodium carboxymethylcellulose, poloxamer, polyethylene glycol, carbomer, methylcellulose, gelatin, xanthan gum, guar gum and amylose starches. In some cases, the polymer is a hydrophilic polymer that is capable of absorbing 100% or more, such as 200% or more, including 500% or more, or 1,000% or more, or 1,500% or more, for instance 2,000% or more of its dry weight in water. In certain embodiments, the polymer absorbs water and swells in volume in an isotropic fashion, although non-isotropic expansions are possible and may be used in certain embodiments. One example of a hydrophilic polymer is aliphatic, polyether-based thermoplastic urethane (TPU). This material is an injection moldable thermoplastic, and may be molded in various shapes, as desired.

In use, the device 400 is positioned within a Eustachian tube 70 in a non-expanded configuration. Water from the patient's body is absorbed into the polymer matrix of driver 410, causing it to gradually expand to the configuration shown in FIG. 21.

Conduit 401 may be made from a metal, a metallic alloy, a polymer, a ceramic or other non-collapsible material and may be configured to constrain the expansion of driver 410 in a direction that is parallel to the axis of device 400, thereby directing the driver 410 to expand substantially in an outward radial direction. For example, the ends of conduit 401 may be flared, creating flanges 403 and 404, which help direct expansion of the driver 410 in an outward radial direction from the conduit 401. Distal flange 404 is optional, and in some embodiments is not present. In some cases, a dilator that does not include distal flange 404 may facilitate insertion of the dilator through the nasopharyngeal opening. Conduit 401 may be flexible. In some cases, conduit 401 is rigid. Conduit 401 may be configured to be non-collapsible under the forces exerted by expanding driver 410 within the confines of Eustachian tube 70, such that conduit 401 reinforces the inner diameter of the device so that the device is capable of exerting force radially outward without collapsing.

Device 400 may be fabricated by an insert molding operation wherein the expandable polymer is molded onto the non-collapsible conduit 401. Additionally, device 400 may be fabricated as two separate parts and joined in a mechanical assembly process to form the final assembled configuration.

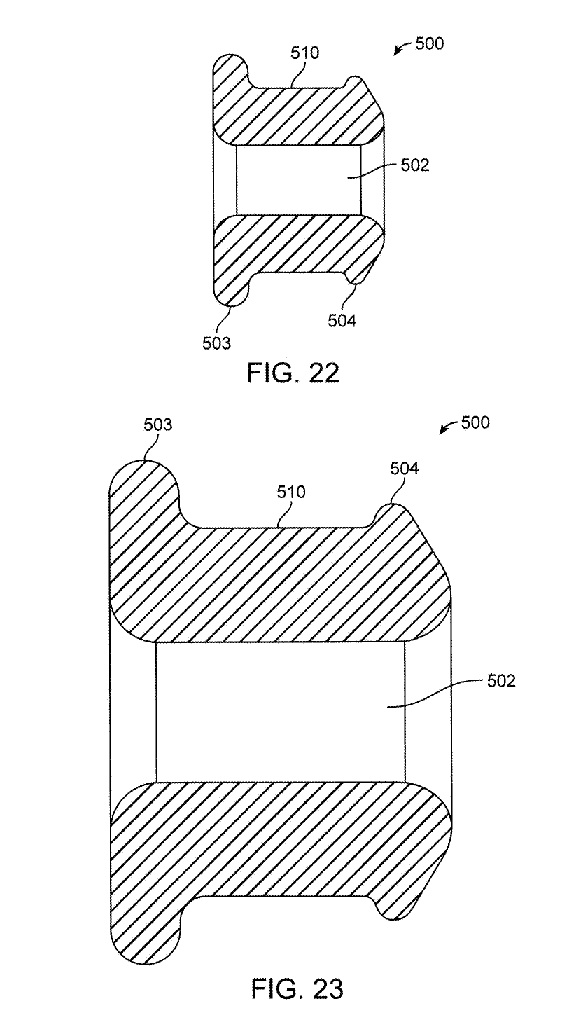

Referring now to FIGS. 22 and 23, there is shown an embodiment of a Eustachian tube dilation device 500 with an expandable driver including a polymer matrix. The interior of device 500 is open, creating an inner conduit 502 for allowing bodily fluids such as mucus, puss and blood to drain out of the middle ear cavity 66 and air to pass into and out of the cavity 66 while the dilation device 500 is positioned within the Eustachian tube 70.

In certain embodiments, the entire device 500 is composed of an expandable polymer matrix 510. The proximal end of matrix 510 is flared, creating anchoring flange 503 which helps keep device 500 from being pushed beyond opening 72 during placement and migrating further into Eustachian tube 70 during use. A suitable expandable polymer for use as matrix 510 is aliphatic, polyether-based thermoplastic urethane (TPU). In certain embodiments, the polymer expands by 100% or more in each linear dimension. The linear expansion may be equivalent to a 700% or more volume expansion. In some instances, the polymer has a specific gravity of between 1.10 and 1.15, which equates to a water absorption of approximately 620% by mass. The matrix material may be an injection moldable thermoplastic, and may be molded in various shapes, as desired. In some instances, the polymer matrix 510 is a homogeneous polymer matrix. In other cases, the polymer matrix 510 is a heterogeneous polymer matrix. For example, the heterogeneous polymer matrix may be configured to have a region of higher rigidity near the interior surface of the device that forms the inner conduit 502. The polymer matrix may also be configured to have regions of higher rigidity at the distal end and the proximal end of the device, such as at the anchoring flanges 503 and 504, respectively. The regions of higher rigidity may facilitate directing the expansion of the polymer matrix radially outward from the inner conduit 502. Distal flange 504 is optional, and in some embodiments is not present. In some cases, a dilator that does not include distal flange 504 may facilitate insertion of the dilator through the nasopharyngeal opening.

In use, the device 500 is positioned within a Eustachian tube 70 in a non-expanded configuration. Water from the surrounding tissues of the patient's body is absorbed into the polymer matrix, causing it to gradually expand to the configuration shown in FIG. 23.

In general, hydrophilic polymeric materials may be manufactured into the configurations shown in FIGS. 20 to 23 by means of injection molding, extrusion, pultrusion, casting, dip coating, spray coating, machining, stereo lithography, selective laser sintering, or any other method suitable for producing the desired geometries.

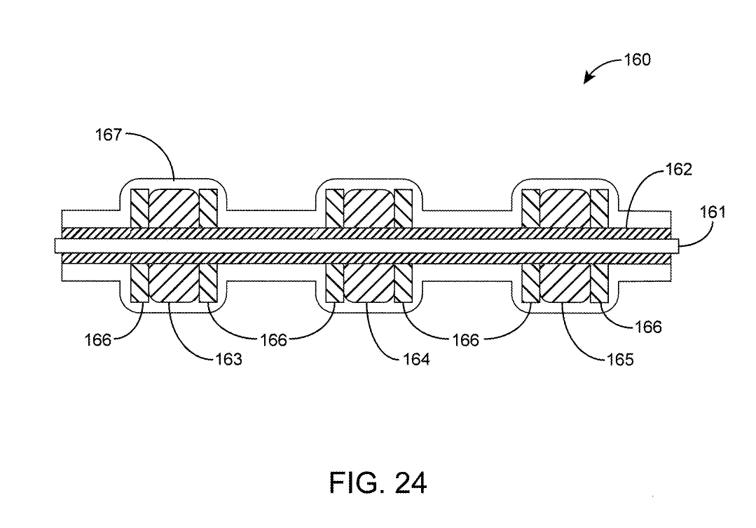

Another embodiment of a Eustachian tube dilator 160 having spaced osmotic tablets 163, 164, 165 is shown in FIG. 24. Central tube 161 is composed of a material that resists collapsing during operation of dilator 160. In one embodiment, the tube is flexible and bendable. On the tube is applied a membrane coating 162 which coating forms a substantially continuous layer over the tube. Osmotic tablets 163, 164, and 165 and microwashers 166 are threaded onto membrane coating 162 such that a microwasher is positioned on both sides of each tablet. Present over the resulting subassembly is rate controlling membrane 167. During the fabrication process, membrane coating 167 and coating 162 are fused such that each osmotic tablet is completely encapsulated by membrane material. The fusion is accomplished by any means to provide a continuous bond between membrane 162 and membrane 167 such as, for example, thermal bonding, solvent bonding, dip coating, and the like.

When in an aqueous environment such as a Eustachian tube, the osmotic dilator 160 imbibes physiological fluids causing radial expansion of the outer elastomeric semipermeable membrane 167. The microwashers serve to direct swelling radially outwardly to further improve Eustachian tube dilation.

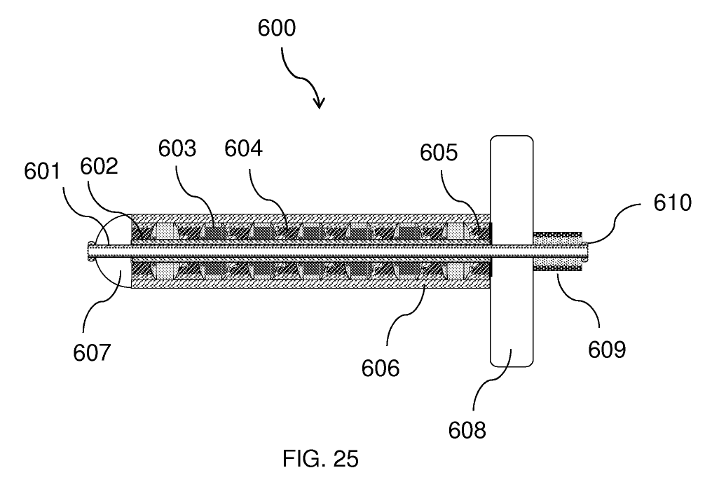

Another embodiment of a Eustachian tube dilator having spaced osmotic tablets is dilator 600 shown in FIG. 25. This dilator comprises a central, flexible conduit 601. Flexible conduit 601 is constructed of thin walled tubing, braided tubing, spiral wound tubing, crisscrossed spiral wound tubing and the like, and may be composed of polymers, metals, or combinations of metals and polymers. Surrounding central conduit 601 is a flexible inner membrane tube 602 having an inside diameter substantially equal to the outside diameter of central conduit 601. The two flexible tubes are therefore in intimate contact and function in concert as a bilayer flexible tube. Osmotic tablets 603, and elastomeric concave spacers 604 and 605, are positioned on the bilayer tube arranged in alternating configuration as illustrated in FIG. 25. A central hole is present in each osmotic tablet such that the inside diameter of the central hole substantially matches the outside diameter of the bilayer tube. Likewise, a central hole is present in each spacer 603 such that the inside diameter of this central hole is substantially equal to the outside diameter of the bilayer tube. Additionally, each osmotic tablet 603 is configured with a convex surface on opposing sides. Each elastic spacer 604 is configured with concave surface on opposing sides. The spacers 604 can be made from elastic materials, such as polymers and rubbers that are easily compressed and/or flexed. In those cases where the dilator 600 is to be inserted for relatively short periods of time (e.g., 4 hours or less), the spacers may be made from materials that exhibit low water absorption so that any water taken up by the osmotic tablets 603 is directed to radial expansion of the dilator 600 and not to saturation of the spacers 604. Suitable materials for spacers 604 include closed cell polyethylene foams, neoprene rubber, and the like. As illustrated in FIG. 25, the tablets and spacers are positioned such that each tablet is placed between two spacers and each tablet and spacer is in intimate contact such that each convex tablet nests between two concave spacers. Spacers 605 positioned at each end of the stack are fabricated with flat faces which flat faces are positioned as illustrated in FIG. 25. A tubular elastic semipermeable membrane 606 is disposed over the tablets 603 and spacers 604. Inside diameter of tubular membrane 606 is substantially equal to the outside diameter of tablets 603 and of spacers 604 and 605.

Inner membrane 602, spacers 604 and 605, and external membrane 606 are processed by chemical or physical means such that the inner membrane is bonded to the spacers and the spacers are bonded to the external membrane. The bonding process may include solvent welding, sonic welding, thermal bonding, dip coating, spray coating and the like. In certain embodiments, inner membrane 602 and external membrane 606 are bonded together at the proximal and distal ends of the dilator. The bonding process may include solvent welding, sonic welding, thermal bonding, dip coating, spray coating and the like.

Dilator 600 also includes a proximal anchor 608 and a proximal anchoring sleeve 609. Proximal anchor 608 has a central hole. Central hole of the proximal anchor 608 is substantially equal to the outside diameter of flexible tube 601. Likewise, the anchoring sleeve 609 has a central hole just slightly larger than the outside diameter of flexible tube 601. Proximal anchor 608 is constructed of flexible material designed to conform to tissues about the nasopharyngeal orifice 72 yet sufficiently sized and sufficiently rigid to block insertion of anchor 608 through the orifice 72 and into the Eustachian tube 70. In certain embodiments, the flex modulus of the proximal anchor material ranges from 5 to 200 mega Pascal, such as 10 to 100 megapascal. Proximal anchoring sleeve is typically constructed of a hard plastic or stainless steel. Proximal anchor 608 is threaded onto flexible tube 601 and bonded using chemical means such as with adhesive. Proximal anchor sleeve 609 abuts the anchor and is attached to flexible tube 601 with chemical or physical means such as screw threads, cyanoacrylate adhesive, laser welding, and the like.

Dilator 600 comprises additionally a distal tip 607. Distal tip 607 has a central hole which is slightly larger in diameter than the outside diameter of flexible conduit 601. Distal tip 607 is constructed of materials as are described for proximal sleeve 609 and is likewise bonded to flexible conduit 601 using chemical or physical means as described herein. The distal tip is sized and shaped to promote ease of insertion and penetration into the Eustachian tube and is configured with a leading face in a bullet, tapered cone, or rounded shape. The device may optionally include flares 610 on both ends of flexible conduit 601 which flares assist to anchor the distal tip 607 and proximal sleeve 609. Flares 610 may be formed by cold forming or thermal forming the ends flexible conduit 601.

During placement of dilator 600 into Eustachian tube 70, the articulated series construction of osmotic tablet 603 and elastomeric spacers 604 provide sufficient flexibility on flexible conduit 601 that the physician can bend and maneuver the device into the anatomy present within the Eustachian tube of a particular patient. Once placed in the Eustachian tube, dilator 600 imbibes water from the biological environment and slowly expands. The outward expansion of the osmotic tablets exerts sufficient force to remodel and expand the diameter of the Eustachian tube.

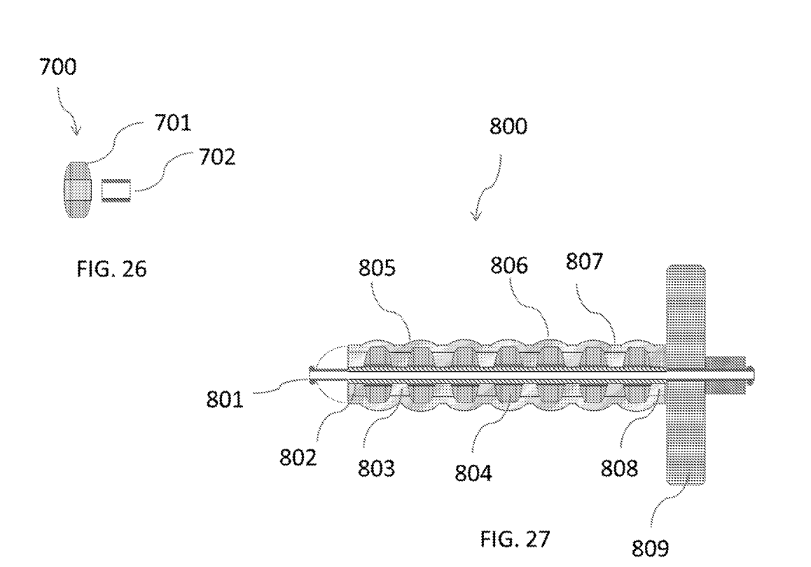

Yet another embodiment of a Eustachian tube dilator 160 having spaced osmotic tablets is dilator 800 shown in FIG. 27 which provides a self-anchoring mechanism to retain the device within the Eustachian tube during the treatment period. Construction and assembly of dilator 800 is similar to construction of dilator 600, with central flexible conduit 801, coated with tubular inner membrane 802, and having proximal and distal flared ends of conduit 101 and proximal anchor 809. Construction of dilator 800 differs from the construction of dilator 600 in that elastomeric spacers 803 are formed with an outside diameter less than the outside diameter of the osmotic tablets 804. Optionally, the osmotic tablets 804 include a two-part subassembly 700 providing an osmotic tablet 701 and tablet sleeve 702, as illustrated in FIG. 26. The osmotic tablet 701 is first formed with a central hole therethrough. Tablet sleeve 702 is formed such that the outside diameter of the sleeve is substantially equivalent to the inside diameter of the central hole of the tablet and the length is substantially equivalent to the length of the central hole within the tablet. Tablet sleeve 702 is then press fit into the center of the tablet to form osmotic tablet assembly 804. The inside diameter of the resulting two-part assembly 804 is substantially equal to the outside diameter of tubular membrane 802 such that two-part osmotic engine assembly 804 can be threaded onto the flexible inner membrane 802.

The central flexible inner membrane 802, spacers 803, spacers 808, and external membrane 805 are processed by chemical or physical means such that the inner membrane 802 of the bilayer tube is bonded to the spacers and the spacers are bonded to the external membrane. In certain embodiments, inner membrane 802 and external membrane 805 are bonded together at the proximal and distal ends of the dilator. This process thereby forms a complete encapsulation of the osmotic tablets. The bonding process may include solvent welding, sonic welding, thermal bonding, dip coating, spray coating, microwave welding, laser welding, and the like. After the bonding process, external tubular membrane 805 undulates over the spacers to create a series of peaks 806 and valleys 807 along the length of the device.

When dilator 800 is inserted by the physician into Eustachian tube 70 of the patient, the dilator is advanced until proximal anchor 809 makes contact with the tissues of nasopharyngeal orifice 72. The ribbed, undulating configuration of dilator 800 with peaks 806 and valleys 807 conforms to the tissue of the Eustachian tube thereby creating an in situ anchoring of the device. Dilator 800 then imbibes water from the biological environment by osmosis, causing the external elastomeric membrane 805 to swell radially. The peaks and valleys enlarge and nest into the tissues to create further anchoring as the diameter of the Eustachian tube is enlarged. At the completion of the treatment period, the physician then retracts the swollen dilator 800.

In yet another application, dilator 800 is fabricated with a central flexible tube 801 which central tube comprises a substrate on the inside surface of tube 801. The substrate is comprised of hydrophobic substance which hydrophobic substance does not wet with water. Central flexible tube 801 comprises the hydrophobic substance. Alternatively, the hydrophobic substance can be applied as a coated layer on the inside surface of tube 801. In another embodiment, the hydrophobic substance is present as a dispersion within the material comprising flexible tube 801.

When dilator 800 with hydrophobic inner tube is placed in the Eustachian tube 70 it imbibes water across the elastomeric membrane and swells to expand the surrounding tissue. The hydrophobic nature of the inner tube does not wet with aqueous biological fluids or cellular matter. Therefore, the central tube 801 provides a conduit for passage of air through the device that facilitates equilibration of air pressure between the middle ear cavity and nasopharynx. Upon insertion of dilator 800, this passage of air provides relief to the patient and maintains the relief throughout and following the treatment period.

Certain sinus ostium dilators are disclosed in FIGS. 3-15 and 20-25 of U.S. patent application Ser. No. 13/219,505, filed Aug. 26, 2011, and also in U.S. Provisional Patent Application, titled “Devices and Methods for Dilating a Paranasal Sinus Opening and for Treating Sinusitis,” filed concurrently with the present application, the disclosures of each of which are incorporated herein by reference in their entirety. Such devices, with appropriate sizing and other modifications including removal of any distal anchors, can also be used in accordance with the methods disclosed herein for dilating a dysfunctional Eustachian tube.

The construction and dimensions, both initial and after dilation, of the dilator may vary depending on the particular Eustachian tube to be dilated and/or the functionality desired. In some cases, the device is constructed according to the physiology of the animal into whose Eustachian tube is intended to receive. As the dilator may be used for any animal having a Eustachian tube, the dilator may be constructed for humans (e.g., patients who are infants, children, teenagers, adults, and seniors), domesticated animals such as dogs, cats, horses, cattle, and pigs, and non-domesticated animals. In the case of a device for dilating a typical adult human

Eustachian tube, the initial (e.g., before expansion/dilation) diameter of the dilator may be 5 mm or less, such as 4 mm or less, including 3 mm or less, or 2 mm or less, or 1 mm or less. For example, the initial diameter of the dilator may range from 1 mm to 5 mm, such as 2 mm to 4 mm, including 2 mm to 3 mm. In some cases, the final (e.g., after expansion/dilation) diameter of the dilator is 4 mm or more, such as 5 mm or more, including 6 mm or more, or 7 mm or more, 8 mm or more, or 10 mm or more. For example, the final diameter of the osmotic driver may range from 2 mm to 10 mm, such as 3 mm to 10 mm, including 4 mm to 8 mm. In certain embodiments, the length of the osmotic driver is 20 mm or less, or 15 mm or less, such as 10 mm or less, including 5 mm or less. For instance, the length of the osmotic driver may range from 3 mm to 20 mm, such as 4 mm to 15 mm, including 5 mm to 10 mm.

For instance, referring back to FIGS. 4 through 7, if conduit 101 has a diameter of 2 mm and the length of the osmotic driver is 10 mm, then the volume of the osmotic core 112 may expand from an initial volume of 0.04 cm³ to a final volume of 0.5 cm³. Accordingly, the semipermeable membrane 111 (assuming the membrane has approximately a cylindrical shape) may be configured to stretch to accommodate the expanding volume of core 112 without breaking. For example, the semipermeable membrane may stretch from an initial surface area of 1 cm² to 3.5 cm². As such, in certain embodiments, the area of the membrane may undergo a 4-fold or more increase in surface area without tearing or rupturing. For example, the membrane may be configured to undergo an approximate 2-fold expansion in each of the X and Y directions. In other words, the membrane may have a 200% or more elongation factor before breaking when stretched in any one axis or direction.

As disclosed herein, in certain embodiments, the rate of expansion of the osmotic driver is such that the driver expands over a period of 0.5 hours or more. Thus, the rate of water imbibition may be such that the dilator expands to the desired size over the desired period of time, e.g., 0.5 hours or more. The rate of volume increase of the osmotic drivers can be approximated by the following equation:

dv/dt=A(k)(Δπ)/L

where:
k is the osmotic water permeability of the semipermeable membrane;
A is the surface area of the semipermeable membrane;
L is the semipermeable membrane thickness; and
Δπ is the osmotic pressure difference across the membrane.

Using this equation, embodiments of the present disclosure may have a rate of volume increase, for example, according to the following: assuming k=9.7×10-6 cm²/hr atm (3.8×10⁻³ cm mil/hr atm); A=0.55 cm²; L=0.038 cm (15 mils); Δπ=356 atm (using NaCl as the osmotic core material) gives an osmotic driver volume increase rate of 0.05 cm³/hour. In certain embodiments, the device has a rate of volume increase ranging from 0.01 cm³/hour to 0.5 cm³/hour, such as 0.05 cm³/hour to 0.45 cm³/hour, including 0.1 cm³/hour to 0.4 cm³/hour, or 0.15 cm³/hour to 0.35 cm³/hour, for example 0.2 cm³/hour to 0.3 cm³/hour.

In some instances, the volumetric imbibition rate is gradually decreased by the buildup of hydrostatic pressure within the osmotic driver as the semipermeable membrane stretches and as the membrane exerts pressure against tissues of the Eustachian tube. When the hydrostatic pressure in the osmotic driver reaches the osmotic pressure of the osmotically active agent within the core, the driver reaches equilibrium and substantially stops expanding. In certain embodiments, the osmotic driver is configured such that the driver reaches equilibrium when the device has expanded to its desired size. In some instances, this provides a safety feature for preventing overexpansion of the surrounding tissues of the patient.

In the above equation, Δπ represents the gradient in osmotic pressure across the semipermeable membrane. The osmotic driving force may depend on the osmotic activity of the mucous layer and other fluids surrounding the Eustachian tube. For example, the π value for normal saline is 8 atm. Therefore, if the osmotic core of the driver contains saturated lactose having a π value equal to 18 atm, and assuming the surrounding mucus has similar activity as saline, then Δπ is 10 atm (18 atm−8 atm=10 atm).

Various semipermeable membranes suitable for human use may be included in embodiments of the osmotic dilators. The polymeric materials from which the semipermeable membranes may be made vary based on the pumping rates and device configuration requirements and include, but are not limited to, plasticized cellulosic materials, enhanced polymethylmethacrylate such as hydroxyethylmethacrylate (HEMA) and elastomeric materials such as polyurethanes and polyamides, polyether-polyamide copolymers, thermoplastic copolyesters and the like. Further semipermeable compositions are described in U.S. Pat. Nos. 5,413,572 and 6,270,787, the disclosures of which are incorporated herein by reference in their entirety. In certain embodiments, the semipermeable membrane material includes cellulose acetate CA398 (Eastman Chemical Co., Kingsport, Tenn.).

In certain embodiments, the semipermeable membranes used in embodiments of the present disclosure also include a plasticizer and/or a rubber-like polymer such as a pharmaceutical grade polyacrylate. One suitable polyacrylate is Eudragit NE30D (Evonik Cyro LLC, Piscataway, N.J.). This material is rubbery and has an elongation at break of 600%, meaning it can be stretched about 6-fold before breaking. Eudragit NE30D serves as a polymeric plasticizer and mixtures of Eudragit NE30D and cellulose acetate CA398 may provide elongation at break (Eb) values that can be tailored to any particular Eustachian tube diameter, with higher Eb values being associated with blends having a higher fraction of NE30D. Elastomers such as silicones can also be used.

The degree of elastic membrane expansion under pressure may depend on membrane thickness, membrane composition, osmotic tablet composition and the shape, configuration and number of the osmotic tablets used. In some instances, the elastic semipermeable membranes exhibit non-uniform expansion. Without being bound to any particular theory, this non-uniformity in membrane expansion may be due to variability in membrane thickness. In other embodiments, the elastic semipermeable membrane expands uniformly. In these embodiments, the elastic semipermeable membrane may have a substantially uniform thickness. When the membranes are applied as multiple coatings of a liquid membrane solution, the membranes may be moved during drying so that thicker coated regions do not develop. For example, an osmotic driver that swells uniformly may include 2 to 4 donut-shaped osmotic tablets formulated with Polyox™ 303 (Dow Chemical Company, Midland, Michigan) and 50 wt % NaCl, together with an expandable semipermeable membrane composed of Tecophilic® HP93A-100 (Lubrizol Corp., Wickliffe, Ohio) coated to a thickness of 0.4 mm (15 mils). These drivers may swell evenly and symmetrically over a period of 4 hours, at which time they reach osmotic equilibrium and substantially stop further swelling, and the symmetry is maintained for 30 hours or more.

As an alternative to a stretchable semipermeable membrane, the membrane may also be composed of a low elongation material that is folded back on itself in the pre-insertion state. For example, the membrane may include materials such as Mylar or polyvinylidene chloride (PVdC). In some cases, the membrane is made to the proper fully expanded size, and then folded upon itself around the osmotic core. In this manner, the membrane unfolds to accommodate the osmotic core as the volume of the osmotic core expands.

Osmotic cores according to embodiments of the present disclosure can include any suitable osmotic agent, examples of which include, but are not limited to, a non-volatile water soluble osmoagent, an osmopolymer which swells on contact with water, or a mixture thereof. Representative osmoagents or osmopolymers are described, for example, in U.S. Pat. Nos. 5,413,572 and 6,270,787, the disclosures of which are incorporated herein by reference in their entirety. Osmotic agents, such as sodium chloride may be used. Sodium chloride in compressed form is an osmotic agent as described, for example, in U.S. Pat. No. 5,728,396, the disclosure of which is incorporated herein by reference in its entirety. The osmotic cores may further include appropriate lubricants, binders, and viscosity modifying agents, such as sodium carboxymethylcellulose or sodium polyacrylate. In certain embodiments, the osmotic agent is capable of generating a pressure ranging from 1 atm to 50 atm, such as 5 atm to 25 atm, including 10 to 20 atm. A summary of suitable osmotic agents (also referred to herein as osmoagents) is listed in Table 1 below. The osmoagents listed in the left column are at saturated concentration in water. The column on the right represents values calculated at one tenth saturated concentration.

### Table 1

### Osmotic Pressures of Various Osmotic Agents

TABLE 1
Osmotic Pressures of Various Osmotic Agents

| | π | | π |
|---|---|---|---|
| Saturated Solute | (atm) | 0.1 Saturated Solute | (atm) |
| lactose-fructose | 500 | lactose-fructose | 50 |
| dextrose-fructose | 450 | dextrose-fructose | 45 |
| urea | 445 | urea | 45 |
| sucrose-fructose | 430 | sucrose-fructose | 43 |
| mannitol fructose | 415 | mannitol fructose | 42 |
| sodium chloride | 356 | sodium chloride | 36 |
| fructose | 355 | fructose | 36 |
| sorbitol | 305 | sorbitol | 31 |
| lactose-sucrose | 250 | lactose-sucrose | 25 |
| lactose-dextrose | 225 | lactose-dextrose | 23 |
| mannitol-dextrose | 225 | mannitol-dextrose | 23 |
| dextrose-sucrose | 190 | dextrose-sucrose | 19 |
| mannitol-sucrose | 170 | mannitol-sucrose | 17 |
| sodium citrate | 165 | sodium citrate | 17 |
| sucrose | 150 | sucrose | 15 |
| citric acid | 150 | citric acid | 15 |
| mannitol-lactose | 130 | mannitol-lactose | 13 |
| dextrose | 82 | dextrose | 8 |
| potassium sulfate | 39 | potassium sulfate | 4 |
| mannitol | 38 | mannitol | 4 |
| sodium phosphate tribasic, | 36 | sodium phosphate tribasic, | 4 |
| 12H2O | | 12H2O | |
| sodium phosphate dibasic, | 31 | sodium phosphate dibasic, | 3 |
| 12H2O | | 12H2O | |
| sodium phosphate dibasic, | 31 | sodium phosphate dibasic, | 3 |
| 7H2O | | 7H2O | |
| sodium phosphate dibasic, | 29 | sodium phosphate dibasic, | 3 |
| anhydrous | | anhydrous | |
| lactose | 18 | lactose | 2 |

Ref:
1) values for saturated solutions from U.S. Pat. No. 4,519,801 except lactose.
2) solubility of lactose from J. Machado, et. al, “Solid-liquid equilibrium of a-lactose in ethanol water”, Phase Equilibria 173 (2000) 121-134. solubility used to calculate osmotic pressure.
3) 0.1 osmotic pressures calculated from van't Hoff law.

The osmotic agent as disclosed in embodiments herein can also be in the form of a polymer. A general description of suitable osmotically active polymers (also referred to herein as osmopolymers) is provided in U.S. Pat. No. 5,160,743, the disclosure of which is incorporated herein by reference in its entirety. Some suitable osmopolymers include, but are not limited to, polyethylene oxide (Polyox® Coagulant Grade, Polyox® 303 low ethylene oxide, Colorcon, Harleysville, Pa.), cellulose gum (Sodium Carboxymethyl Cellulose Grade 7H4F, Aqualon, Wilmington, Del.), and polyacrylic acids (Carbopol® Grades 974 NF, EDT2020 NF, Ultrez 10 NF, and ETD 2020NF, Lubrizol Corporation, Wickliffe, Ohio).

In certain embodiments, at least portions of the dilation devices as disclosed herein are formed of bioerodible (also referred to herein as bioabsorbable) materials that are capable of breaking down and either being absorbed by, or expelled by, the patient's body. Such bioerodible or bioabsorbable materials include metals, polymers, and bioactive glasses. Suitable bioerodiblebioabsorbable metals include magnesium alloys, including formulations such as the magnesium alloys disclosed in U.S. Patent Application No. 2002/0004060, the disclosure of which is incorporated herein by reference in its entirety. In some instances, the bioerodiblebioabsorbable alloy includes 50-98% magnesium, 0-40% lithium, 0-5% iron and 5% or less of other metals. Other suitable formulations include a magnesium alloy having 90% or more magnesium, 3.7%-5.5% yttrium, and 1.5%-4.4% rare earths. Additional formulations are disclosed in U.S. Patent Application No. 2004/0098108, the disclosure of which is incorporated herein by reference in its entirety. Suitable bioerodiblebioabsorbable polymers include polylactic acid, polyglycolic acid, collagen, polycaprolactone, hylauric acid, adhesive protein, co-polymers of these materials, as well as composites and combinations thereof.

In certain embodiments the entire dilation device is formed of bioerodible/bioabsorbable materials. In these embodiments, no active removal of the device is required, e.g., the device is passively removed through the process of bioerosion/bioabsorption. In some instances, only a portion of the device is composed of bioerodible materials. For example, the drug reservoirs may include bioerodible/bioabsorbable material. In these embodiments, drug releasing bioerodible/bioabsorbable polymers can be used, including those disclosed in U.S. Pat. Nos. 5,464,450; 6,387,124; and 5,500,013, the disclosures of which are incorporated herein by reference in their entirety.

In another embodiment, methods are provided for inserting a dilator into a Eustachian tube of an animal. The method involves inserting the dilator as described above through a nostril or mouth of the animal into the Eustachian tube. The device may be inserted solely through the nasopharyngeal opening of the Eustachian tube through the nose or the oropharynx in a manner that does not involve making any incision to a tympanic membrane or ear canal skin. The device may then be released in a manner effective to allow the device to immobilize itself within the Eustachian tube at its opening in the nasopharynx. The method may be performed with local anesthesia or sedation as appropriate.

Depending on the particulars of the nasal cavity configuration, the device may be inserted into the mouth or which ever nostril that allows for greater ease for device placement in either the right or left Eustachian tube. In particular, the device may be placed through the nasal passages or through the oropharynx under the palate. Advantageously, the method does not require an incision in the ear canal or tympanic membrane, or entry into the middle ear space.

Once the dilator has been properly positioned and immobilized, the Eustachian tube remains substantially unobstructed. During the dilator-induced patency of the Eustachian tube, the middle ear is aerated. The device effectively becomes a portal to drain fluid and infection from the middle ear.

Removing the device from the Eustachian tube may include use of the same access routes that were used for dilator insertion. Removing the device can be done using forceps to grab the proximal anchor 105, 125, 157, 609 or 809. Alternatively, a string or loop attached to the dilator can be provided, similar to loop 703 shown in FIG. 20 of U.S. patent application Ser. No. 13/219,505, filed Aug. 26, 2011, the disclosure of which is incorporated herein by reference in its entirety.

The dilator insertion method may optionally be carried out through endoscopy, in conjunction with surgery or in the absence of any incision. Regardless whether the Eustachian tube into which the device is inserted is surgically unaltered or altered, the method may be effective to temporarily dilate the Eustachian tube. Furthermore, the method may be effective to prevent collapse of the Eustachian tube and/or involve insertion of the device into an enlarged Eustachian tube.

The disclosed methods and devices can be used to treat certain types of hearing loss, tinnitus, ear discomfort and headache. The method can treat dysfunction of the Eustachian tube due to scarring from surgery, radiation treatment, infection and inflammation affecting the Eustachian tube. The device also enables diagnostic microendoscopy of Eustachian tube and the middle ear, and serves as a conduit for the diagnosis and assessment of middle and inner ear functions, integrity of the ossicles, chronic ear infection and cholesteatoma. Further, the device serves as a stent and protective dressing for any hard and soft palate, nasopharyngeal, or Eustachian tube surgery.

Still further, the dilators may be effective to treat retracted tympanic membranes and ear congestion. By dilating the Eustachian tube, allergic and/or infectious Eustachian tube dysfunction may also be treated. Both chronic and acute Eustachian tube dysfunction may be treated.

Aspects of the present disclosure include a system for dilating a dysfunctional Eustachian tube in a subject. The systems include a device for dilating the Eustachian tube and an insertion device configured to position the device in the Eustachian tube. The device may include an expandable portion configured to expand from a non-expanded configuration to an expanded configuration, and a driver configured to expand the expandable portion from the non-expanded configuration to the expanded configuration, as described herein.

Suitable insertion devices are described herein and also in U.S. patent application Ser. No. 13/219,497, filed on Aug. 30, 2011, and also in U.S. Provisional Patent Application, titled “Devices and Methods for Dilating a Paranasal Sinus Opening and for Treating Sinusitis,” filed concurrently with the present application, the disclosures of each of which are incorporated herein by reference in their entirety.

In certain embodiments, the system includes a device for dilating a dysfunctional Eustachian tube and a stent. The stent may be configured such that the device fits within the stent when the device is in a non-expanded configuration, as described herein. For example, the stent may have a cylindrical shape with a diameter that is slightly greater than the diameter of the device when the device is in a non-expanded configuration. In some cases, the stent is an expandable stent. The expandable stent may be configured to expand in size as the device expands from a non-expanded configuration to an expanded configuration. In certain embodiments, the stent is configured to maintain its expanded configuration after it has been expanded from the non-expanded configuration to the expanded configuration. For example, the stent may be configured, such that the stent is able to expand from a non-expanded configuration to an expanded configuration, but upon application of a force to the exterior surface of the stent, may maintain substantially the same interior diameter or deform under application of the force and then return to substantially the same interior diameter after removal of the external force. In some cases, the stent may be configured such that pressure exerted on the exterior surface of the stent by the surrounding tissues during use does not significantly decrease the interior diameter of the stent. A stent that is configured to maintain its expanded configuration may facilitate dilation of the Eustachian tube. The stent may be made of any suitable material, such as a shape-memory alloy, including but not limited to nitinol, stainless steel, titanium, cobalt-chromium alloy, combinations thereof, and the like.

As discussed above, an insertion apparatus may be used to place the dilator into and/or extract the dilator from the Eustachian tube. The insertion apparatus may have any of a number of designs and construction. The insertion apparatus, in some embodiments, is endoscopic and hand held in construction. The apparatus should provide a user sufficient degree of control over the insertion and/or extraction of the dilator in a minimally invasive manner so as to minimize trauma or discomfort to a patient. Thus, the apparatus may provide for precisely and accurately controlled translational (e.g., X-Y-Z) and/or, rotational (.theta.-.phi.) movement capabilities. The dilator insertion apparatus may allow for one, two, three, four, five, six, or more degrees of freedom.

For example, the insertion apparatus may have a dilator-interfacing terminus and a manipulation terminus. The dilator-interfacing terminus may have a construction specific to the dilator or may be used to interface with devices other than those described herein. For example, the interfacing terminus may have a solid or hollow geometry specific to the dilator. In some instances, the interfacing terminus may also provide for functionality associated with the practice of the methods described herein. Exemplary functionalities include suction, aspiration, delivery of air or medications to the middle ear.

The manipulation terminus may house a means for releasing any dilator engaged therewith. The releasing means may have a spring-loaded mechanism, or manual release mechanism that allows the dilator to be releasably engaged with device-interfacing terminus of the apparatus. Optionally, the dilator may be controllably slid from the insertion apparatus into the Eustachian tube.

In some instances, the dilator may be constructed with a means for interfacing with the insertion apparatus. In some instances, such means serve no other purpose than to interface with the insertion apparatus. For example, the interfacing means may include at least one protrusion extending from an exterior surface by which the insertion apparatus may mount or grab. As another example, one or more tabs or fenestrations may be located on the proximal end of the dilator.

In the alternative, the interfacing means may serve a plurality of purposes. For example, the fluid-communication providing means may have a construction effective to serve as means for engaging with the insertion apparatus. When a hole is provided as the fluid-communication providing means, the insertion apparatus may be constructed to engage the device via the fluid-communication-providing means through a friction fitting.

As another alternative, the interfacing means may be used to make adjustments to the device to be inserted and/or extracted. For example, the interfacing means may be used to adjust the fluid-communication providing means. When the fluid-communication providing means is in the form of a central passageway, the passageway may be made smaller or larger through the insertion device.

The dilator may be packaged with the insertion apparatus to form a kit. Typically, the kit also includes container for containing the insertion apparatus and the dilator. Optionally, instructions for using the insertion apparatus and the dilator may be included.

Aspects of the present disclosure additionally include kits that have a device for dilating a dysfunctional Eustachian tube in a subject, as described in detail herein. The kits may include one or more dilation devices, where the devices may be provided in a variety of different sizes. The size of the device may depend on the physiology of the subject to be treated, the severity of dysfunctionality and/or stenosis of the Eustachian tube, etc. Additional embodiments of the kits may include a drug, such as, but not limited to an antibiotic, an anti-inflammatory drug, anesthetics (e.g., local anesthetics), analgesics (e.g., locally acting analgesics), vasoconstrictors, combinations thereof, and the like. The drug may be provided in a separate container, such as a syringe, vial, bottle, etc., such that the drug may be filled into the drug reservoir of the device prior to insertion of the device into the Eustachian tube.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., one or more pieces of paper on which the information is printed, in the packaging of the kit, in a package insert, etc. Another form would be a computer readable medium, e.g., diskette, CD, DVD, Blu-Ray, computer-readable memory, etc., on which the information has been recorded or stored. Yet another form of providing instructions to a user may be a website address which may be used via the Internet to access the information at a removed site. Any convenient means of providing instructions may be present in the kits.

The length of time during which the dilator may remain in place within the Eustachian tube may be selected according to a plurality of selection criteria, singly and in combination. For example, when the device is constructed to treat a disorder associated with the Eustachian tube, the rate of dilator expansion may be chosen to ensure restoration of proper Eustachian tube function within a predetermined period without having trauma to the Eustachian tube mucosa that potentially may lead to scarring. In instances where the Eustachian tube dysfunction is minor and the patient is only suffering from the discomfort and/or hearing loss due to middle ear pressure being unequal with ambient air pressure, the period of time during which the dilator resides within the Eustachian tube may be less that 4 hours, less than 2 hours and even less than 1 hour.

When placed in the Eustachian tube at the torus tubarius, e.g., through a minimally invasive procedure that results in device placement through the nasal passages or through the oropharynx under the palate, the device confers a number of advantages previously unknown in the art. For example, the benefits of a nasopharyngeal-based therapy may be achieved without the disadvantages of the undesirable outcomes associated with treatment methods that involve an incision in the ear canal or tympanic membrane, or entry into the middle ear space. In addition, such placement of the device in the Eustachian tube may provide immediate relief from fluid in the ear and pressure related maladies.

As the device renders the Eustachian tube substantially unobstructed, the device may aerate the middle ear, become a portal to drain fluid and infection from the middle ear, treat retracted tympanic membranes and ear congestion, and the like. The device may also enable diagnostic microendoscopy of Eustachian tube and the middle ear, and serve as a conduit for the diagnosis and assessment of middle and inner ear functions, integrity of the ossicles, chronic ear infection and cholesteatoma.

As can be appreciated from the disclosure provided above, the present disclosure has a wide variety of applications. Accordingly, the following examples are offered for illustration purposes and are not intended to be construed as a limitation on the invention in any way. Those of skill in the art will readily recognize a variety of noncritical parameters that could be changed or modified to yield essentially similar results. Thus, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### EXAMPLES

### Example 1

A bendable, osmotic dilation system for treating Eustachian tube dysfunction is fabricated. 304 stainless steel tubing having an outside diameter of 0.020 inch and inside diameter of 0.012 inch and wall thickness of 0.004 inch is cut to lengths of 20 mm. The tubing is supplied by Small Parts Incorporated, Logansport, Ind. Next, an extruded polyurethane tube having an outside diameter of 0.036 inch, inside diameter of 0.020 inch, and length of 14.4 mm is slipped onto the stainless steel tube and positioned on the tube such that 1.8 mm of bare metal is present at one end and 3.8 mm of bare metal is present at the opposite end. The polyurethane tube comprises a uniform blend of 9 parts Tecophilic HP93A-100 and 1 part polyvinyl pyrrolidone. The Tecophilic is supplied by Lubrizol, Wilmington, Mass., and the polyvinyl pyrrolidone is supplied as Kollidon 12PF by BASF Corporation, Ludwigshafen, West Germany. This completes subassembly of the flexible, subcoated tube.

An osmotic engine composition is prepared. 8.5 grams of polyoxyethylene, 15.0 grams of sodium chloride, and 1.25 grams of hydroxypropyl methylcellulose are passed through a 100 mesh sieve into a beaker and mixed with a spatula to form a uniform blend. The polyoxyethylene is supplied by Colorcon, West Point, Pa., as Polyox™ WSR 303, the sodium chloride USP grade is supplied by Sigma-Aldrich, St. Louis, Mo., and the hydroxypropyl methylcellulose is supplied as Methocel™ E5 by Dow Chemical Co., Midland, Mich. 7 ml of anhydrous ethanol, formula SD3A, is then stirred slowly into the dry mixture to form a uniformly damp mass. The damp mass is next forced with a spatula through a 40 mesh sieve to form extruded segments. The extruded segments are dried in a force air oven overnight at 40° C. Then, the dried segments are passed again through a 40 mesh sieve, to form free-flowing granules. Finally, 0.25 grams of magnesium stearate is passed through an 80 mesh sieve over the granules and tumble mixed into the granular blend for 2 minutes. This completes the osmotic granulation.

A batch of minitablets is compacted from the osmotic granulation using with a Carver press fitted with core rod tooling. The core rod tooling has an outside diameter of 2.6 mm and an inside diameter of 0.92. Configuration of the tablet punch faces is standard concave round tooling, nominal compression force is 60 pounds, and nominal weight of the tablets is 12 mg. This completes fabrication of the osmotic engines.

Biconcave elastomeric discs for use between osmotic engines are injection molded of 9 parts Tecophilic HP93A-100 and 1 part Kollidon 12PF. Diameter of the discs is 2.6 mm. The disc is configured with concave faces on both sides such that the thickness on the outside edge is 1.44 mm and thickness in the center is 0.96 mm. Concavity is configured to match the convex surfaces of the tablet faces such that each disc nests evenly between the faces of two osmotic engines. The biconcave elastomeric disc has a central hole with a diameter of 0.92 mm that matches the diameter of the central hole of the tablets. Elastomeric discs for use at the ends of the device are also injection molded with the same composition and configuration as above except that one face is concave and one face of the disc is flat.

Osmotic engines and elastomeric discs are threaded onto the polyurethane tube. Seven tablets and 6 elastomeric biconcave discs are threaded onto the tube such that one elastomeric disc is positioned between each osmotic engine and one elastomeric disc with one flat face is threaded onto each end.

An extruded tube of polyurethane having an inside diameter of 2.6 mm, and outside diameter of 3.26 mm and length of 14.4 mm, is slipped over the section of the device having the stack of engines and discs such that one end of the device has 1.8 mm or bare metal exposed and the other end has 3.8 mm of bare metal exposed.

A stainless steel, domed-shape tip with central hole is attached to the 1.8 mm bare metal end using medical grade cyanoacrylate adhesive. The adhesive is supplied as Loctite 4011 from Loctite Corporation, Rocky Hill, Conn. Diameter of the dome is 3.1 mm, the length is 1.3 mm, and diameter of the central hole is 0.023 inch. This forms the distal tip of the device.

The proximal end of the device is next fitted with a proximal anchor comprising low-durometer polymer stamped from 1.7 mm sheet stock. Configuration of the proximal anchor is in the form of a dog bone and includes a central hole having a diameter of 0.022 inch. The anchor is threaded onto the exposed 3.8 mm bare metal end. A stainless steel sleeve having a length of 1.7 mm, an outside diameter of 1.7 mm, and an inside diameter of 0.023 inch is threaded onto the bare metal and glued with the same medical grade adhesive. Both ends of the stainless steel tubing are mechanically flared to a diameter of 0.026 inch using a tapered flaring tool.

The device is subjected to a current of heated air which heat is conducted into the device, thereby melting and bonding the internal polyurethane tube residing on the stainless steel tube to the polyurethane discs and also melting and bonding the polyurethane discs to the external polyurethane membrane. Thus, each osmotic engine is fully encapsulated by the same polyurethane composition. This completes fabrication of the device.

The resulting dilation device is inserted by an ear nose and throat physician through the nasal cavity of a patient and inserted into the Eustachian tube. The proximal anchor is sized larger than the opening of the Eustachian tube and therefore prevents the device from being inserted too far into the duct. Given the small diameter and thin wall of the stainless steel tube, the device can be bent to such an extent that it during insertion, it conforms to the slight natural curvature of the Eustachian tube. The segmented construction of alternating non-flexible tablets and flexible and compressible elastomeric discs provide sufficient pliability for insertion but prevent over bending of the device which over bending may kink the steel tube causing blockage of the central the stainless steel tube. By maintaining patency of the tube of the device, air pressure can equilibrate during the treatment period between the middle ear and the nasal cavity, providing immediate relief to the patient followed by continuous relief throughout the treatment period. Once in place, the device continuously imbibes water by osmosis from tissues of the patient. As the elastomeric rate controlling membrane slowly expands from an initial diameter of 3.1 mm to a final diameter of 5 mm over a period of 1 hour, the tissue of the Eustachian tube is gradually enlarged and remodeled by the expanding device. After the treatment period, the dilation device is removed. The Eustachian tube, now remodeled, is restored to normal and healthy biological function, capable of continuously opening and closing such that it can now maintain equal air pressure between the middle ear and the nasal cavity resulting in continuous and long-term relief for the patient.

### Example 2

A self-anchoring, flexible osmotic stent for treating Eustachian tube dysfunction is fabricated. A flexible polyimide tubing having an outside diameter of 0.020 inch, inside diameter of 0.014, and wall thickness of 0.003 inch is cut into lengths of 80 mm. The polyimide tubing is supplied by Small Parts Company, Plainsfield, Ill. An extrude tube comprising a polyurethane composition and having an inside diameter of 0.020 inch, outside diameter of 0.028 inch, and wall thickness of 0.004 inch is slipped over the polyimide tube. The polyurethane tube comprises 95 parts Tecophilic HP93A-100 and 5 parts polyvinyl pyrrolidone. The Tecophilic is supplied by Lubrizol, Wilmington, Mass., and the polyvinyl pyrrolidone is supplied as Kollidon 17PF by BASF Corporation, Ludwigshafen, West Germany. This completes subassembly of the flexible, subcoated tube.

A batch of osmotic engines is fabricated using the same composition and processing conditions as describe in Example 1. A batch of sleeves are cut into lengths of 0.050 inch from 304 stainless steel tubing having an inside diameter of 0.028 inch and an outside diameter of 0.036 inch. The sleeve are next inserted into the inside diameter of the osmotic tablets.

A batch of elastomeric biconcave discs comprising 95 parts Tecophilic HP93A-100 and 5 parts Kollidon 17PF was injection molded. Configuration of the discs was as described in Example 1 except that the outside diameter of the disc was 2.0 mm rather than 2.6 mm.

Seven osmotic engines having sleeves, and eight elastomeric discs were threaded onto the polyurethane tube using the positioning described in Example 1 such that the convex face of each 2.6 mm diameter engine was abutted to a convex face of a 2.0 mm diameter elastomeric disc. Proximal and distal anchors were then attached as described in Example 1. An extruded tube of polyurethane having an inside diameter of 2.6 mm, and outside diameter of 3.26 mm and length of 14.4 mm, is slipped over the section of the device having the stack of engines and discs. Composition of this external tube was 95 parts Tecophilic HP93A-100 and 5 parts Kollidon 17PF. Finally, the device is subjected to a current of heated air which heat is conducted into the device, thereby melting and bonding the internal polyurethane tube residing on the polyimide tube to the polyurethane discs and also melting and bonding the polyurethane discs to the external polyurethane membrane. Thus, each osmotic engine is fully encapsulated by the same polyurethane composition. Additionally, during the thermal process, the external membrane conforms to the 2.6 mm tablets and 2.0 mm discs to form a ribbed configuration with seven peaks and eight valleys spanning the working length of the device. This completes fabrication of the device. Overall length of the device is 20.0 mm.

The device is placed by a physician by way of the nasal cavity into the Eustachian tube of a patient using the procedures described in Example 1. The flexibility of the articulated structure on the flexible inner polyimide tube allows the surgeon to bend the device such that it conforms to the curvature of the Eustachian tube. Additionally, the ribbed configuration of the device provides a sufficiently smooth surface for ease of insertion while the undulating geometry conforms to the live tissue to thereby lodge and anchor the device pressing upon the tissues. As the device imbibes water from the tissues of the patient and the osmotic engines enlarge radially from an initial diameter of 3.3 mm to a final diameter of 5 mm over a one hour period, tissue of the Eustachian tube is expanded and remodeled. The stainless steel sleeves of the osmotic engines prevent the polyimide tube from collapsing during expansion of the engines. Additionally, the undulating configuration of the device mated with the remodeled, undulating tissue provides a continuous anchoring surface to prevent premature dislodgement of the device into the nasal cavity from the trumpet-shaped opening of the Eustachian tube.

### Example 3

A device for treating Eustachian tube dysfunction which device remains patent of mucous, water, blood, and aqueous fluids during operation is manufactured.

The device is fabricated according to the compositions and procedures described in Example 2 except that the polymer inner tube is polyimide that has a hydrophobic surface lining the lumen of the tube. When the device is in operation, aqueous body fluids do not wet the inner surface of the tube due to the hydrophilic nature of the surface. As a result, the polyimide tube remains substantially unobstructed during treatment such that air can freely pass through the center of the device and allow equilibration of air pressure between the inner ear and the nasal cavity.

### Example 4

A device for treating Eustachian tube dysfunction is manufactured according the procedures and compositions of Example 1 except that the solid wall stainless steel tubing is replaced with braided wall stainless steel tubing. The braided wall tubing imparts sufficient flexibility to allow the device to conform to the gentle curvature of the Eustachian tube yet also provides sufficient resistance to buckling under pressure expansion of the osmotic engines.

### Example 5

A device for treating Eustachian tube dysfunction is manufactured according to the procedures and compositions of Example 2 except that five osmotic engines are used instead of seven. Overall length of the device is 16.1 mm.

### Example 6

A device for treating Eustachian tube dysfunction is manufactured according to the procedures and compositions of Example 2 except that four osmotic engines are used instead of seven. Overall length of the device is 14.1 mm.

### Example 7

A device for treating Eustachian tube dysfunction is manufactured according to the procedures and compositions of Example 2 except that three osmotic engines are used instead of seven. Overall length of the device is 12.3 mm.

### Example 8

A device for treating Eustachian tube dysfunction is manufactured according to the procedures and compositions of Example 2 except that a thin coating of surfactant is applied to the surface of the external membrane. After the treatment period and after the device is removed from the Eustachian tube, a temporary residue remains on the surface of the soft tissues, which residue assists in preventing these tissues from adhering to each other so that the Eustachian tube can freely open and close during the subsequent few hours of tissue restoration after treatment.

### Example 9

A medical device for treating Eustachian tube dysfunction over a 24 hour period is manufactured according to the procedures and compositions described in Example 2 except that the subcoat membrane, elastomeric discs, and overcoat membrane are comprised of Tecoflex Grade EG100A. This thermoplastic polyurethane polymer is supplied by Lubrizol, Cleveland, Ohio.

### Example 10

A medical device for treating Eustachian tube dysfunction over a 48 hour period is manufactured according to the procedures and compositions described in Example 9 except that the overcoat membrane is comprised of 0.026 inch Tecoflex Grade EG100A.

### Example 11

A medical device for treating Eustachian tube dysfunction over a 7-day period is manufactured according to the procedures and compositions described in Example 10 except that the overcoat membrane is comprised of a bilayer coating. The bilayer coating comprises a first applied layer of 0.015 inch of Tecoflex grade EG100A. The second applied layer comprises 0.005 inch ethyl acrylate methylmethacrylate 70:30 copolymer supplied as Eudragit® NM by Evonik Industries, Darmstadt, West Germany.

### Example 12

A device for treating Eustachian tube dysfunction is manufactured according the procedures and compositions of Example 1 or 2 except that the solid wall stainless steel tubing is replaced with a laser spiral cut hypotube. The spiral cut hypotube will be supplied by Creganna-Tactx, Galway, Ireland. The spiral cuts of the hypotube are patterned in a fashion that provides sufficient flexibility with minimal axial elongation or compression. This flexibility allows the device to conform to the curvature required to access the Eustachian tube while providing sufficient column strength to aid in pushability. The minimal axial elongation will provide sufficient rigidity to ensure expansion from the osmotic engines is driven in the radial direction. The cut width will be less than 50% of total component surface area to provide sufficient resistance to buckling under pressure expansion of the osmotic engines.

### Example 13

A device for treating Eustachian tube dysfunction is manufactured according the procedures and compositions of Example 1 or 2 except that the solid wall stainless steel tubing is replaced with a flat stacked coil welded radially on both ends 360 degrees. The coil is made from a flat ribbon wire and is coiled in a stacked configuration with a pitch angle of 0-70 degrees from the axial axis. The coil configuration provides sufficient flexibility with minimal axial elongation or compression. This flexibility allows the device to conform to the curvature required to access the Eustachian tube while providing sufficient column strength to aid in pushability. The minimal axial elongation provides sufficient rigidity to ensure expansion from the osmotic engines is driven in the radial direction. The cut width is less than 50% of total component surface area to provide sufficient resistance to buckling under pressure expansion of the osmotic engines. The coil is supplied by Precision Wire Components Tualatin, Oreg.

### Example 14

A flexible dilator with segmented construction for use in treating Eustachian tube dysfunction is manufactured. Pieces of 304 stainless steel tube stock having an inside diameter of 0.032 inch (0.081 cm), an outside diameter of 0.042 inch (0.11 cm) and a length of 55 mm are dip coated in an elastomeric semipermeable membrane coating solution comprising a 10 wt % solids solution of polyurethane (Tecophilic grade HP60D-20; Thermedics™ Polymer Products, Wilmington, Mass.) dissolved in n-methyl pyrrolidone. The tubes are dip coated multiple times until a membrane coating having a nominal coating thickness of 0.005 inch (0.01 cm) accumulates on the middle of each of the tubes. The tubes are dried in a current of room temperature air between coatings. Polyether ether ketone polymer stock is machined to form microwashers having an inner opening diameter of 0.055 inch (0.14 cm), an outside diameter of 0.110 inch (0.28 cm) and a thickness of 0.020 inch (0.05 cm). The average weight of the microwashers is 3 mg. Three osmotic salt-containing tablets and six microwashers are then threaded onto the coated stainless steel tubes such that a microwasher is placed in contact with each tablet, forming three distinct sets of microwasher+salt tablet+microwasher sandwiched subassemblies. Additionally, a 1.5 mm gap is provided between the middle and the end subassemblies. The tubes with subassemblies are dip coated multiple times in the same membrane coating solution until a continuous elastomeric semipermeable membrane coating on the salt tablets develops. Between dip coatings, the dilators are dried in a current of room temperature air. A proximal anchor is optionally attached to the proximal end of the tube.

When in an aqueous environment such as a Eustachian tube, the osmotic dilator imbibes physiological fluids causing radial expansion of the outer elastomeric semipermeable membrane. The microwashers serve to direct swelling radially outwardly to further improve Eustachian tube dilation.

The preceding merely illustrates the principles of the disclosure. All statements herein reciting principles, aspects, and embodiments of the disclosure as well as specific examples thereof, are intended to encompass both structural and functional equivalents thereof. Additionally, it is intended that such equivalents include both currently known equivalents and equivalents developed in the future, e.g., any elements developed that perform the same function, regardless of structure. The scope of the present disclosure, therefore, is not intended to be limited to the exemplary embodiments shown and described herein. Rather, the scope and spirit of present disclosure is embodied by the appended claims.

That which is claimed is:

1. A method of dilating a dysfunctional Eustachian tube of an animal, the method comprising:
inserting a self-expanding dilator into the Eustachian tube through a nasopharyngeal opening of the Eustachian tube via an oral or nasal passageway of the animal,
wherein the dilator is configured to be self-expanding after insertion and includes a driver configured to expand an expandable portion from a non-expanded configuration to an expanded configuration, wherein the non-expanded configuration is sized to be positioned within the dysfunctional Eustachian tube and the expanded configuration has a size sufficient to dilate the Eustachian tube.

2. The method of claim 1, wherein the self-expanding dilator expands over a period of 0.5 hours or more.

3. The method of claim 2, wherein the self-expanding dilator expands over a period of 0.5 to 4 hours.

4. The method of claim 2, wherein the self-expanding dilator expands over a period of 0.5 to 2 hours.

5. The method of claim 1, wherein the driver comprises an osmotically active agent.

6. The method of claim 1, wherein the driver comprises an expandable polymeric matrix.

7. The method of claim 1, wherein the driver is configured to absorb water from the animal to expand the dilator.

8. The method of claim 1, comprising removing the dilator after the expandable portion expands from the non-expanded configuration to the expanded configuration.

9. The method of claim 1, wherein the dilator is configured to equalize pressure within a middle ear of the animal with ambient pressure.

10. The method of claim 1, comprising draining liquid from a middle ear cavity of the animal while the dilator is in the Eustachian tube.

11. The method of claim 1, wherein the animal is a human.

12. A dilator for dilating a dysfunctional Eustachian tube of an animal, the dilator comprising:
a self-expanding dilator configured to expand an expandable portion from a non-expanded configuration to an expanded configuration, wherein the non-expanded configuration is sized to be inserted through a nasopharyngeal opening of the Eustachian tube via an oral or nasal passageway of the animal and positioned within the dysfunctional Eustachian tube,
wherein the dilator is configured to be self-expanding after insertion and includes a driver configured to expand an expandable portion from a non-expanded configuration to an expanded configuration, wherein the expanded configuration has a size sufficient to dilate the Eustachian tube.

13. The dilator of claim 12, wherein the self-expanding dilator expands over a period of 0.5 hours or more.

14. The dilator of claim 13, wherein the self-expanding dilator expands over a period of 0.5 to 4 hours.

15. The dilator of claim 13, wherein the self-expanding dilator expands over a period of 0.5 to 2 hours.

16. The dilator of claim 12, wherein the driver comprises an osmotically active agent.

17. The dilator of claim 12, wherein the driver comprises an expandable polymeric matrix.

18. The dilator of claim 12, wherein the driver is configured to absorb water from the animal to expand the dilator.

19. The dilator of claim 12, comprising a retaining anchor configured to retain the dilator in the Eustachian tube while the expandable portion expands from the non-expanded configuration to the expanded configuration.

20. The dilator of claim 12, wherein the dilator is configured to be removed from the Eustachian tube after the expandable portion expands from the non-expanded configuration to the expanded configuration.

21. The dilator of claim 12, comprising a fluid passageway extending entirely through the dilator.

22. The dilator of claim 21, wherein the passageway is configured to equalize pressure within a middle ear of the animal with ambient pressure.

23. The dilator of claim 21, wherein the passageway is configured to drain liquid from a middle ear cavity of the animal.

24. The dilator of claim 12, wherein the dilator is sized and shaped to be inserted in, and dilate, a dysfunctional Eustachian tube of a human.

25. The dilator of claim 12, wherein the dilator has a flexural modulus of 4,300 psi or less.

26. A kit comprising the dilator of claim 12 and instructions for the use thereof.

27. The kit of claim 26, comprising an insertion device configured to insert the dilator into the Eustachian tube of the animal.
